(19) United States
(12) Patent Application Publication
(10) Pub. No.: US 2022/0259768 A1
(43) Pub. Date: Aug. 18, 2022

(54) MULTIVALENT CHLOROTOXIN CHIMERIC ANTIGEN RECEPTORS

(71) Applicant: IN8bio, Inc., New York, NY (US)

(72) Inventors: Lei Ding, Vestavia Hills, AL (US); Lawrence S. Lamb, JR., Birmingham, AL (US)

(21) Appl. No.: 17/580,199

(22) Filed: Jan. 20, 2022

Related U.S. Application Data
(60) Provisional application No. 63/139,709, filed on Jan. 20, 2021.
(60) Provisional application No. 63/172,247, filed on Apr. 8, 2021.

(51) Int. Cl. C40B 40/02

(52) U.S. Cl. CPC C40B 40/02; C07K 2317/622; C07K 2317/24; C07K 2317/21

(57) ABSTRACT

Described are γδ T-cells that express a multivalent CLTX-CAR and also express a survival factor, a population of the γδ T-cells that express a multivalent CLTX-CAR and the survival factor, pharmaceutical compositions thereof, and methods of treating cancer or a tumor in a subject comprising administering to a subject an effective amount of the multivalent CLTX-CAR γδ T-cells and co-administering a chemotherapeutic agent, e.g., the chemotherapeutic agent to which the survival factor confers resistance.

### RELATED APPLICATIONS

This application claims the benefit of U.S. Provisional Application No. 63/139,709 filed Jan. 20, 2021 and U.S. Provisional Application No. 63/172,247 filed Apr. 8, 2021. The entire contents of the above applications are incorporated herein by reference.

### BACKGROUND OF THE INVENTION

Chimeric antigen receptors (CARs) are composed of an extracellular tumor recognition/targeting domain, an extracellular linker/hinge domain, a transmembrane domain, and intracellular T-cell-activating and co-stimulatory signaling domains. The majority of CAR tumor targeting domains are single chain variable fragments (scFvs) derived from antibody sequences that exploit the specificity of antibody binding to particular antigens. For example, the anti-CD19 targeted CARs KYMRIAH™, and YESCARTA™ are approved therapies for the treatment of acute lymphoblastic leukemia (ALL) and diffuse large B-cell lymphoma, respectively, and comprise scFvs derived from a murine anti-human CD19 antibody (Guedan et al. (2019), Mol Ther Methods Clin Dev 12:145-156).

WO2018107134 and WO2017066481, the contents of each of which are expressly incorporated by reference herein, describe chimeric antigen receptors comprising chlorotoxin (CLTX) as the extracellular antigen binding domain. CLTX is a small natural peptide derived from scorpion venom that specifically targets altered expression of matrix metalloproteinase 2 (MMP2) and chloride channel CLCN3 on glioblastoma multiforme (GBM). CLTX also binds melanoma, small cell lung carcinoma, neuroblastoma, breast cancer, kidney cancer, liver cancer, lung cancer, ovarian cancer, and medulloblastoma among others. The primary structure of chlorotoxin comprises 36 amino acids including eight cysteines and is classified as a short-chain, disulfide containing peptide. WO2018107134 specifically describes gamma delta (γδ) T-cells engineered to express chlorotoxin.

Gamma-delta (γδ) T-cells are an important subset of T lymphocytes as they can recognize a broad range of antigens without antigen priming or the presence of major histocompatibility complex (MHC) molecules. They can target and kill cells directly through their cytotoxic activity or indirectly through the activation of other immune cell types. γδ T-cell functional responses are induced by several factors including the recognition of stress antigens, which promotes cytokine production and regulates pathogen clearance, inflammation, and tissue homeostasis in response to stress (e.g., a chemotherapeutic agent environment). The cytotoxicity of γδ T-cells to tumors can be induced through the expression of cell surface receptors, including natural killer group 2D ligand (NKG2DL), on tumor cells.

While recent advances in immunotherapies have shown promise in treating extracranial tumors, GBM has remained impervious to these advances with a consistent median survival of less than 15 months. There remains a need in the art for additional treatments, and specifically additional CAR T-cell therapies, for GBM as well as other liquid and solid cancers and tumors.

### SUMMARY OF THE INVENTION

The present invention is based, at least partially, on the surprising discovery that γδ T cells transduced with a CAR comprising two CLTX peptides in the extracellular antigen-binding domain demonstrate increased persistence as well as increased cytotoxicity against glioblastoma (GBM) cells as compared to comparable cells with a single CLTX peptide. The increased cytotoxicity was observed even when the cells did not include an intracellular signaling domain within the endodomain. In addition, it is shown that transduction of T cells with more than two CLTX peptides (e.g., three of more CLTX peptides) did not enhance activation and resulted in a CAR that is not properly or functionally presented on the cell surface. In addition, the invention is at least partially based on the surprising discovery that T-cells transduced with a CAR comprising two CLTX peptides in the extracellular antigen-binding domain (dCLTX-CAR cells) demonstrated greater than 2-fold CD69 activation and as compared to cells comprising a single CLTX peptides in the extracellular antigen binding domain (sCLTX-CAR γδ T-cells). While it may have been predicted that the presence of two CLTX peptides would be additive and result in greater, e.g., two-fold greater, CD69 activation than a single CLTX peptide, it is surprising that the presence of only two CLTX peptides in the CAR resulted in more than 2 times greater activation than a comparable CAR with a single CLTX peptide. This data suggests that the presence of multiple CLTX peptides (two CLTX peptides) in the extracellular antigen binding domain has a synergistic effect on T cell activation and unexpectedly shows greater persistence and greater cytotoxicity against glioblastoma cells.

The invention encompasses γδ T-cells that express a multivalent CLTX-CAR (a CAR that comprises more than one CTLX peptide in the extracellular antigen-binding domain) and also express a survival factor, wherein the survival factor is a DNA, an RNA or a polypeptide that confers resistance to a chemotherapeutic agent, a population of the γδ T-cells that express a multivalent CLTX-CAR and the survival factor, pharmaceutical compositions comprising the multivalent CLTX-CAR γδ T-cells, and methods of treating cancer or a tumor in a subject comprising administering an effective amount of the multivalent CLTX-CAR γδ T-cells and co-administering a chemotherapeutic agent, e.g., the chemotherapeutic agent to which the survival factor confers resistance. The multivalent CLTX-CAR preferably comprises two CLTX peptides in the extracellular antigen-binding domain. A CLTX-CAR that includes two CLTX peptides can be referred to herein as a “divalent CLTX-CAR” or a “dual CLTX-CAR” or a “dCLTX-CAR” (these terms are used interchangeably herein). In certain aspects, the multivalent CLTX-CAR (e.g, the dCLTX-CAR) does not comprise an intracellular signaling domain.

The invention includes an engineered γδ T-cell that expresses a multivalent CLTX chimeric antigen receptor (a multivalent CLTX-CAR) and a survival factor, wherein the survival factor is a DNA, RNA or polypeptide that confers resistance to a chemotherapeutic agent, wherein the γδ T-cell comprises a single vector that directs the expression of the multivalent CLTX-CAR and the survival factor, and further wherein:

a. the multivalent CLTX-CAR comprises:
i. an extracellular antigen-binding domain comprising at least two of the following peptides: a CLTX peptide, a CLTX-like polypeptide, and a functional variant of CLTX peptide (including combinations thereof),
   wherein the at least two CLTX peptides, CLTX-like polypeptides, and functional variants of CLTX peptide, or a combination of any of thereof, are attached by a linker peptide; optionally the linker peptide is less than 30 amino acids in length, or 15 amino acids in length;
ii. a transmembrane domain;
iii. an optional extracellular hinge domain that attaches the transmembrane domain to the extracellular antigen-binding domain;
iv. an optional intracellular signaling domain; and
v. optionally, a co-stimulatory domain. In certain aspects, multivalent CLTX-CAR comprises two peptides selected from the group consisting of a CLTX peptide, a CLTX-like polypeptide, a functional variant of CLTX peptide, and a combination thereof.

In specific embodiments, the invention is directed to an engineered γδ T-cell that expresses a multivalent CLTX chimeric antigen receptor (a multivalent CLTX-CAR) and a survival factor, wherein the survival factor is a polypeptide that confers resistance to a chemotherapeutic agent, wherein the γδ T-cell comprises a single vector that directs the expression of the multivalent CLTX-CAR and the survival factor, and further wherein:

a. the multivalent CLTX-CAR comprises:
i. an extracellular antigen-binding domain comprising at least two CLTX peptides,
   wherein the at least two CLTX peptides are attached by a linker peptide; optionally the linker peptide is less than 30 amino acids in length, or 15 amino acids in length;
ii. a transmembrane domain;
iii. an optional extracellular hinge domain that attaches the transmembrane domain to the extracellular antigen-binding domain;
iv. optionally, an intracellular signaling domain; and
v. optionally, a co-stimulatory domain.

The invention also encompasses a population of the engineered γδ T-cells described herein. In certain aspects, multivalent CLTX-CAR comprises two CLTX peptides in the extracellular antigen-binding domain. In certain aspects, the multivalent CLTX-CAR (e.g, the dCLTX-CAR) does not comprise an intracellular signaling domain. In certain additional aspects, the linker peptide is a Flag peptide, a myc peptide or an HA peptide.

In yet additional aspects, the invention is directed to an engineered γδ T-cell that expresses a divalent CLTX chimeric antigen receptor (or a divalent CLTX CAR) and a survival factor, wherein the survival factor is a polypeptide that confers resistance to a chemotherapeutic agent, wherein the γδ T-cell comprises a single vector that directs the expression of the divalent CLTX-CAR and the survival factor, and further wherein:
a. the divalent CLTX-CAR comprises:
   i. an extracellular antigen-binding domain comprising two CLTX peptides, wherein the two CLTX peptides are attached by a linker peptide;
   optionally the linker peptide is less than 30 amino acids in length, or 15 amino acids in length;
   ii. a transmembrane domain;
   iii. an optional extracellular hinge domain that attaches the transmembrane domain to the extracellular antigen-binding domain;
   iv. an optional intracellular signaling domain; and
   v. an optional co-stimulatory domain.

The invention also encompasses a population of the engineered γδ T-cells described herein. In certain specific aspects, the divalent CLTX CAR comprises a co-stimulatory domain and does not include an intracellular signaling domain. In further aspects, the divalent CLTX CAR does not include an intracellular signaling domain and does not include a co-stimulatory domain. In yet further aspects, the linker peptide is a Flag peptide, a myc peptide or an HA peptide.

The invention additionally includes a pharmaceutical composition comprising the multivalent CLTX-CAR γδ T-cells (or a population of the multivalent CLTX-CAR γδ T-cells) as described herein as well as a method of treating cancer or tumor in a subject in need thereof, the method comprising administering to said subject a composition comprising the multivalent CLTX-CAR γδ T-cells as described herein and co-administering to said subject an effective amount of the chemotherapeutic agent; for example, the effective amount is an amount sufficient to increase stress antigen expression on the cancer or tumor cells. Also encompassed is a pharmaceutical composition comprising the divalent CLTX-CAR γδ T-cells (or a population of the divalent CLTX-CAR γδ T-cells) as described herein as well as a method of treating cancer or tumor in a subject in need thereof, the method comprising administering to said subject a composition comprising the divalent CLTX-CAR γδ T-cells as described herein and co-administering to said subject an effective amount of the chemotherapeutic agent; for example, the effective amount is an amount sufficient to increase stress antigen expression on the cancer or tumor cells.

In certain aspects, the survival factor is a polypeptide that confers resistance to a chemotherapeutic agent. The polypeptide that confers resistance to a chemotherapeutic agent can, for example, be selected from the group consisting of alkyl guanine transferase (AGT), O⁶ methylguanine DNA methyltransferase (MGMT), P140K MGMT (also referred to herein as MGMTp140k), L22Y-DHFR, thymidylate synthase, dihydrofolate reductase, multiple drug resistance-1 protein (MDR1), 5′ nucleotidase II, dihydrofolate reductase, and thymidylate synthase. The polypeptide can, for example, can refer resistance to any chemotherapeutic agent, for example an alkylating agent. In additional aspects, the polypeptide confers resistance to a chemotherapeutic agent selected from the group consisting of trimethotrexate, temozolomide, raltitrexed, S-(4-Nitrobenzyl)-6-thioinosine, 6-benzyguanidine, nitrosoureas, fotemustine, cytabarine, and camptothecin.

The multivalent CLTX-CAR or divalent CLTX-CAR can additionally express a suicide gene. A non-limiting example of a suicide gene is thymidine kinase, for example, the herpes simplex virus thymidine kinase (HSV-TK).

The multivalent CLTX-CAR can comprise two CLTX peptides, three CLTX peptides, four CLTX peptides, or more than four CLTX peptides. In certain aspects, the multivalent CLTX-CAR comprises two CLTX peptides. In additional aspects, the multivalent CLTX-CAR comprises two CLTX peptides, three CLTX peptides, or four CLTX peptides and the survival factor is MGMT or MGMTp140k. In yet further aspects, the multivalent CLTX-CAR comprises comprise two CLTX peptides, and the survival factor is MGMT. In certain aspects, the CLTX-CAR is a dCLTX-CAR, the survival factor is MGMT or MGMTp140k, and the dCLTX-CAR does not include an intracellular signaling domain. In further aspects, the CLTX-CAR is a dCLTX-CAR, the survival factor is MGMT or MGMTp140k, and the dCLTX-CAR comprises a co-stimulatory domain (e.g., CD28 co-stimulatory domain) and does not include an intracellular signaling domain. In yet additional aspects, the CLTX-CAR is a dCLTX-CAR, the survival factor is MGMT or MGMTp140k, and the dCLTX-CAR does not include an intracellular signaling domain and does not include a co-stimulatory domain.

The multivalent CLTX-CAR can comprise a transmembrane domain comprising a CD28 transmembrane domain; and/or an intracellular signaling domain comprises the CD3 zeta (also referred to herein as CD3z or CD3ζ) signaling domain; and/or a hinge domain that comprises the hinge region of a protein selected from the group consisting of CD8, CD28, and/or CD137. In certain aspects, the co-stimulatory domain is present and comprises the CD28 co-stimulatory domain and/or the 4-1BB co-stimulatory domain. The multivalent CLTX-CAR can additionally comprise an extracellular signal peptide; for example, the signal peptide is the signal peptide of a protein selected from the group consisting of CD8, CD28, GM-CSF, CD4, CD137, or a combination thereof. Exemplary linker peptides are c-myc, FLAG, and (GSSS)ₙ.

The multivalent CLTX-CAR or divalent CLTX-CAR can comprise a transmembrane domain comprising a CD28 transmembrane domain; and/or a hinge domain that comprises the hinge region of a protein selected from the group consisting of CD8, CD28, and/or CD137. The multivalent or divalent CLTX-CAR can additionally comprise an extracellular signal peptide; for example, the signal peptide is the signal peptide of a protein selected from the group consisting of CD8, CD28, GM-CSF, CD4, CD137, or a combination thereof. The multivalent or divalent CLTX CAR can comprise a linker peptide; exemplary linker peptides are c-myc, FLAG, HA, and (GSSS)ₙ.

In certain embodiments, the multivalent CLTX-CAR does not comprise or include an intracellular signaling domain. In further aspects, the multivalent CLTX-CAR does not comprise or include a CD3 zeta signaling domain. In additional embodiments, the multivalent CLTX-CAR comprises a co-stimulatory domain (e.g., CD28 co-stimulatory domain) and does not comprise a CD3 zeta signaling.

Also specifically encompassed is a divalent CLTX-CAR, wherein the endodomain does not comprise or include an intracellular signaling domain. In further aspects, the endodomain of the divalent CLTX-CAR does not comprise or include a CD3 zeta signaling domain. In additional embodiments, the divalent CLTX-CAR does comprise a co-stimulatory domain (e.g., CD28 co-stimulatory domain) and does not comprise a CD3 zeta signaling. Also encompassed is a divalent CLTX-CAR that does not comprise a CD3 zeta signaling and does not comprise a co-stimulatory domain (e.g., CD28 co-stimulatory domain).

The methods of treatment can, for example, be for the treatment of an intracranial tumor. The tumor can, for example, be a glioma such as glioblastoma. In certain aspects the multivalent CLTX-CAR γδ T-cells or a composition thereof are administered intracranially. The stress antigen expressed by the tumor cells in response to the chemotherapeutic agent can be an NKG2DL (NKG2D ligand), for example. Non-limiting examples of NKG2DLs included, but are not limited to, MIC-A, MIC-B, ULBP-1, ULBP-2, ULBP-3 and ULBP-4. The multivalent CLTX-CAR γδ T-cells, specifically including divalent CLTX-CAR γδ T-cells, can have enhanced cytotoxicity to the tumor cells as compared to that of comparable sCLTX-CAR gamma delta T-cells. In further aspects, the multivalent CLTX-CAR γδ T-cells, specifically divalent CLTX-CAR γδ T-cells, can have enhanced persistence as compared to that of comparable sCLTX-CAR γδ T-cells. A “comparable sCLTX-CAR” is identical to the multivalent CLTX-CAR γδ T-cell to which it is compared to except that it comprises a single CLTX peptide in the antigen recognition domain. A composition comprising the comparable sCLTX-CAR is identical to that of the multivalent CLTX-CAR γδ T-cell to which it is being compared (e.g., the number of cells is the same; the excipients are the same, the mode of administration is the same, etc.).

In certain aspects, the multivalent CLTX-CAR γδ T-cell or a composition thereof has enhanced cytotoxicity to tumor cells in the chemotherapeutic agent environment (to which the survival factor confers resistance) than a comparable sCLTX-CAR γδ T-cell or composition thereof. In additional aspects, the multivalent CLTX-CAR γδ T-cell or a composition thereof has enhanced (e.g., CD69 activation) activation as compared to that of a comparable sCLTX-CAR γδ T-cell or composition thereof. For example, a divalent CLTX-CAR γδ T-cell or a composition thereof can have enhanced cytotoxicity to tumor cells in the chemotherapeutic agent environment (to which the survival factor confers resistance) than a comparable sCLTX-CAR γδ T-cell or composition thereof. In an additional aspect, the divalent CLTX-CAR γδ T-cell does not include an intracellular signaling domain, or a composition thereof, and has enhanced cytotoxicity as compared to a comparable sCLTX-CAR γδ T-cell or composition thereof. In a further aspect, the divalent CLTX-CAR γδ T-cell comprises a co-stimulatory domain (e.g., a CD28 co-stimulatory domain) and does not include an intracellular signaling domain, or a composition thereof, and has enhanced cytotoxicity as compared to a comparable sCLTX-CAR γδ T-cell or composition thereof. In yet an additional aspect, the divalent CLTX-CAR γδ T-cell does not comprise a co-stimulatory domain (e.g., a CD28 co-stimulatory domain) and does not comprise an intracellular signaling domain and has enhanced cytotoxicity as compared to a comparable sCLTX-CAR γδ T-cell or composition thereof.

In further aspects, the multivalent CLTX-CAR γδ T-cell, or a composition thereof, has enhanced persistence as compared to a comparable sCLTX-CAR γδ T-cell or composition thereof. For example, a divalent CLTX-CAR γδ T-cell or a composition thereof has enhanced persistence as compared to a comparable sCLTX-CAR γδ T-cell or composition thereof. In an additional aspect, the divalent CLTX-CAR γδ T-cell does not include an intracellular signaling domain and has enhanced persistence as compared to a comparable sCLTX-CAR γδ T-cell or composition thereof. In a further aspect, the divalent CLTX-CAR γδ T-cell does not include an intracellular signaling domain, and has enhanced persistence as compared to a comparable divalent CLTX-CAR γδ T-cell that does include an intracellular signaling domain, for example, a CD3z signaling domain. In yet a further aspect, the divalent CLTX-CAR γδ T-cell comprises a co-stimulatory domain (e.g., a CD28 co-stimulatory domain) and does not include an intracellular signaling domain and has enhanced persistence as compared to a comparable divalent CLTX-CAR γδ T-cell that does include an intracellular signaling domain, for example, a CD3z signaling domain. In additional embodiments, the divalent CLTX-CAR γδ T-cell does not comprise a co-stimulatory domain (e.g., a CD28 co-stimulatory domain) and does not comprise an intracellular signaling domain and has enhanced persistence as compared to a comparable divalent CLTX-CAR γδ T-cell that does include an intracellular signaling domain, for example, a CD3z signaling domain.

The invention additionally encompasses methods of enhancing the cytotoxicity of CLTX-CAR γδ T-cells to tumor cells in a subject undergoing treatment with a chemotherapeutic agent, the method comprising engineering the γδ T-cells to express at least two CLTX peptides and a survival factor as described herein, wherein the multivalent CLTX-CAR γδ T-cells (or composition thereof) have enhanced cytotoxicity as compared to comparable sCLTX-CAR γδ T-cells (or composition thereof), and further comprising administering the engineered γδ T-cells to the subject. In yet additional aspects, the invention encompasses methods of increasing the activation (e.g., CD69 activation) of CLTX-CAR gamma delta T-cells to tumor cells in a subject undergoing treatment with a chemotherapeutic agent, the method comprising engineering the gamma delta T-cells to express at least two CLTX peptides and a survival factor, wherein the multivalent CLTX-CAR γδ T-cells (or composition thereof) display enhanced activation as compared to comparable sCLTX-CAR γδ T-cells (or composition thereof), and further comprising administering the engineered γδ T-cells to the subject. In certain specific aspects, the method comprises engineering the γδ T-cells to express two CLTX peptides (a divalent CLTX-CAR) and a survival factor as described herein, wherein the divalent CLTX-CAR γδ T-cells (or composition thereof) have enhanced cytotoxicity as compared to comparable sCLTX-CAR γδ T-cells (or composition thereof). In certain embodiments, the divalent CLTX-CAR does not include an intracellular signaling domain. In yet further aspects, the divalent CLTX-CAR comprises a co-stimulatory domain (e.g., a CD28 co-stimulatory domain) and does not include an intracellular signaling domain. In additional aspects, the divalent CLTX-CAR does not comprise a co-stimulatory domain (e.g., a CD28 co-stimulatory domain) and does not comprise an intracellular signaling domain.

The invention further includes methods of enhancing the persistence of CLTX-CAR γδ T-cells in a subject undergoing treatment with a chemotherapeutic agent, the method comprising engineering the γδ T-cells to express at least two CLTX peptides and a survival factor as described herein, wherein the multivalent CLTX-CAR γδ T-cells (or composition thereof) have enhanced persistence as compared to comparable sCLTX-CAR γδ T-cells (or composition thereof), and further comprising administering the engineered γδ T-cells to the subject. In certain aspects, the multivalent CLTX-CAR γδ T-cells comprise a signaling domain. In yet further aspect, the multivalent CLTX-CAR do not comprise a signaling domain. In additional aspects, the multivalent CLTX-CAR do not comprise a CD3z signaling domain. In certain specific aspects, the method comprises engineering the γδ T-cells to express two CLTX peptides (a divalent CLTX-CAR) and a survival factor as described herein, wherein the divalent CLTX-CAR γδ T-cells (or composition thereof) have enhanced persistence as compared to comparable sCLTX-CAR γδ T-cells (or composition thereof). In certain embodiments, the divalent CLTX-CAR does not include an intracellular signaling domain. In yet further aspects, the divalent CLTX-CAR comprises a co-stimulatory domain (e.g., a CD28 co-stimulatory domain) and does not include an intracellular signaling domain. In additional aspects, the divalent CLTX-CAR does not comprise a co-stimulatory domain (e.g., a CD28 co-stimulatory domain) and does not comprise an intracellular signaling domain.

The invention additionally includes a nucleic acid or vector encoding the multivalent CLTX-CAR or a divalent CLTX-CAR as described herein. In certain aspects, the nucleic acid or vector further encodes a survival factor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing a dual CLTX-CAR (dCLTX-CAR) in a cell that expresses MGMT. The two CLTX peptides of the dCLTX-CAR are linked by a c-myc (“Myc”) peptide linker. Also shown are the hinge region, the transmembrane region, the intracellular co-stimulatory domain, and the intracellular signaling domain.

FIG. 2 is a construct map of exemplary CLTX-CAR constructs. Starting from the left, the CLTX-CAR constructs can optionally comprise a CD8 leader sequence (“CD8L”), one or two CLTX peptides (e.g., CLTX and “Null,” and CLTX and CLTX, respectively) linked by a Myc or Flag peptide, a CD8 hinge region (“CD8H”), a CD28 transmembrane domain (“CD28tm”), a CD28 co-stimulatory domain (“CD28co”), a CD3 zeta signaling domain (“CD3ζ” or “Z”) or no signaling domain (“noZ”), a P2A peptide, and MGMT or EGFP. The depicted CLTX-CAR constructs include the following dCLTX (dual CLTX peptides) constructs:

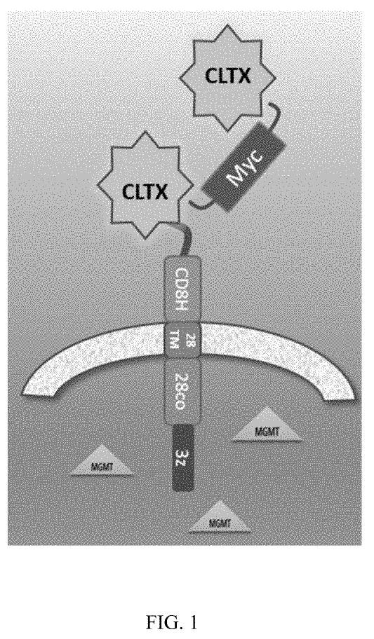

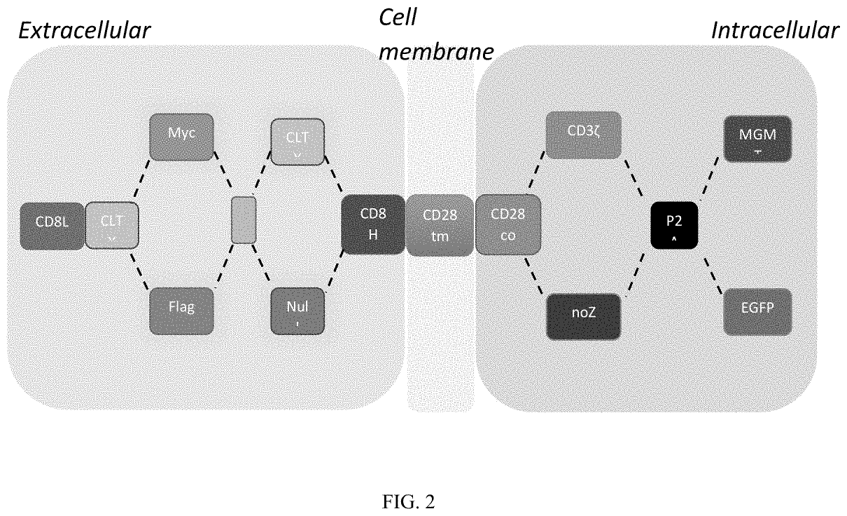

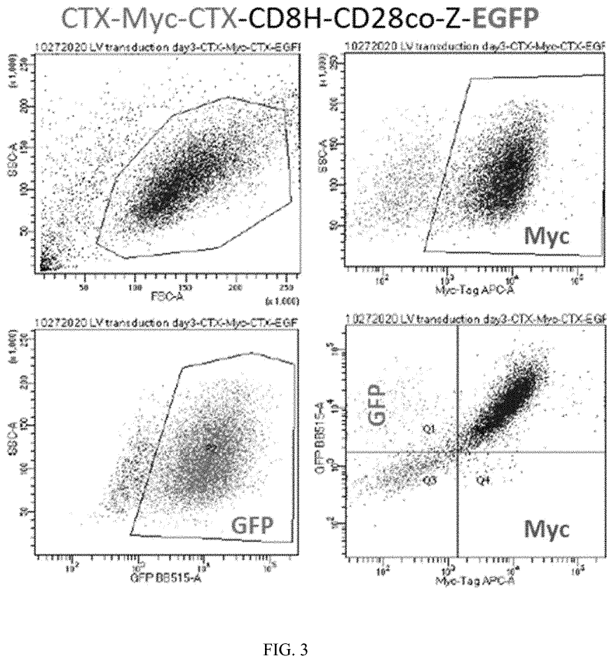

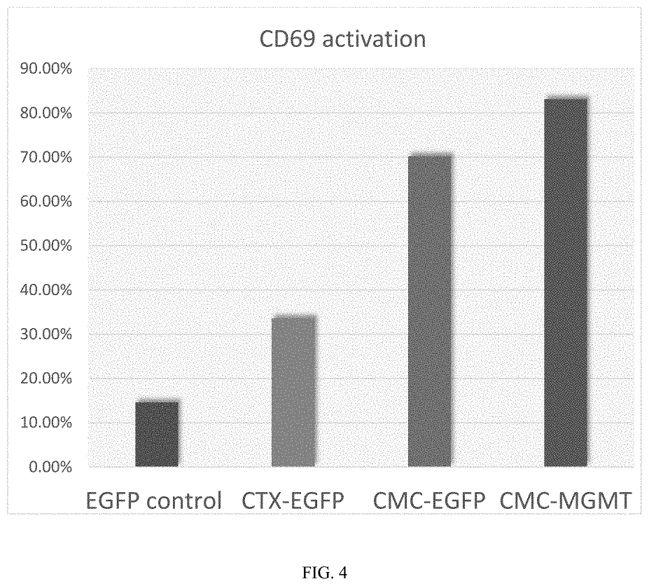

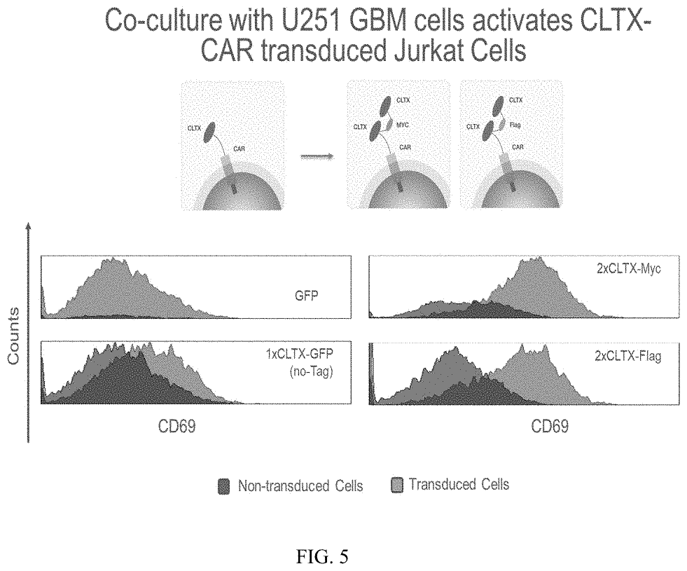

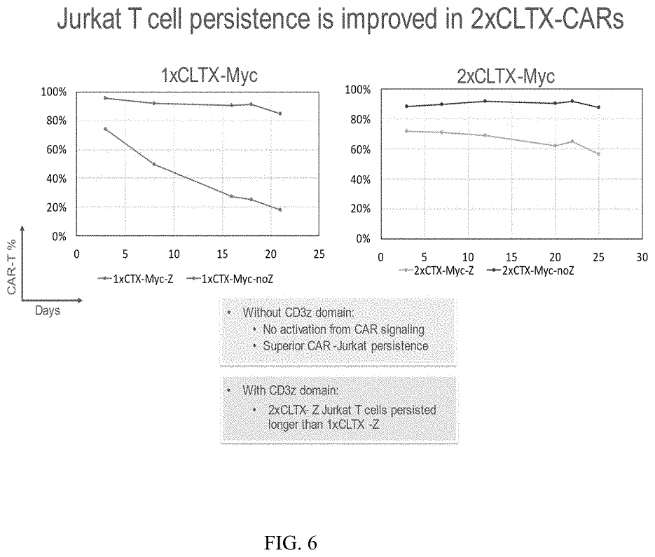

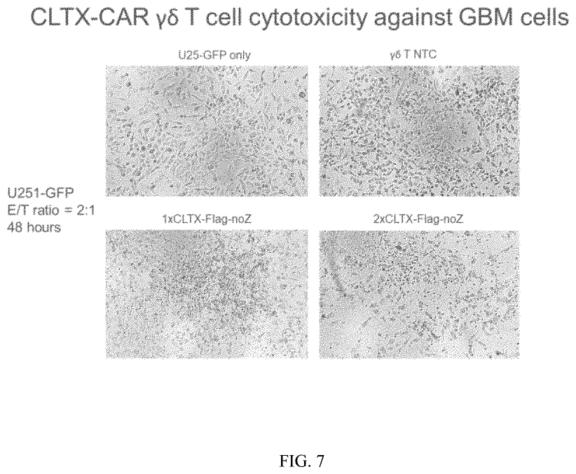

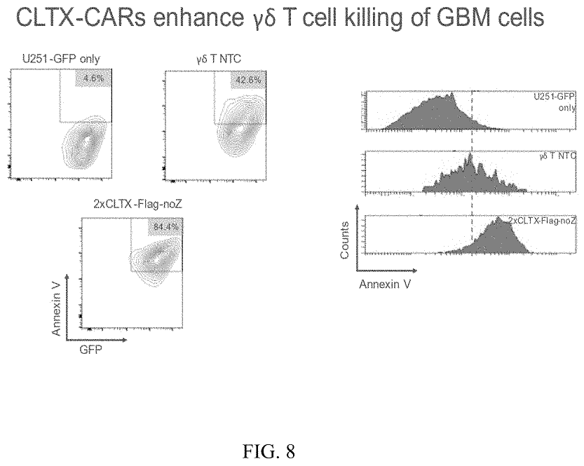

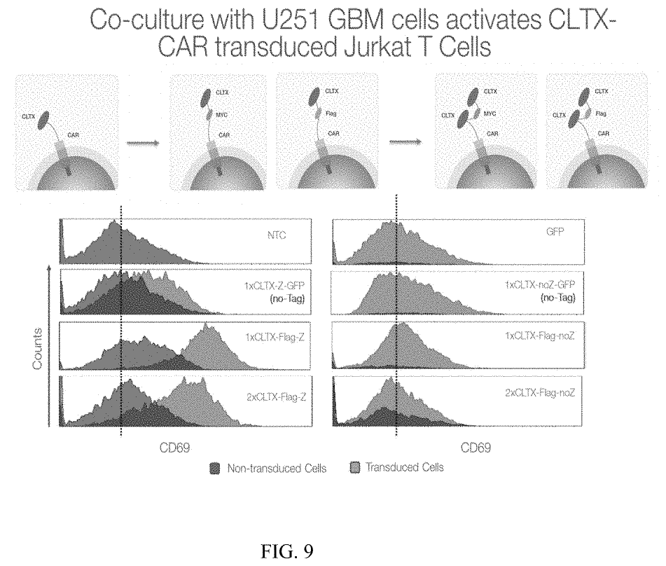

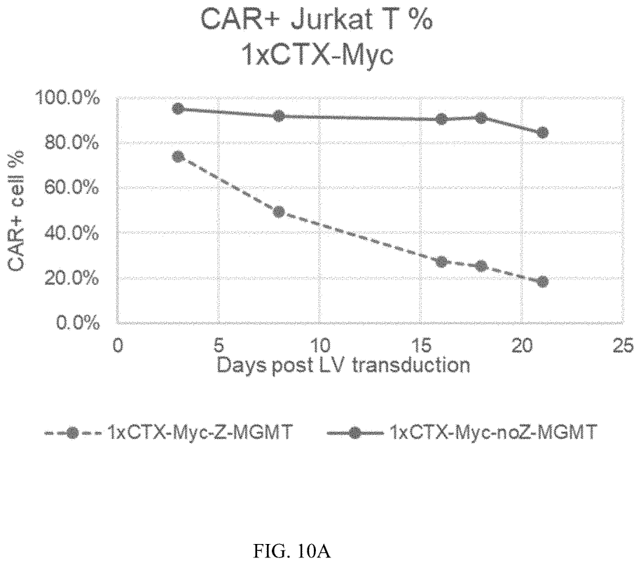

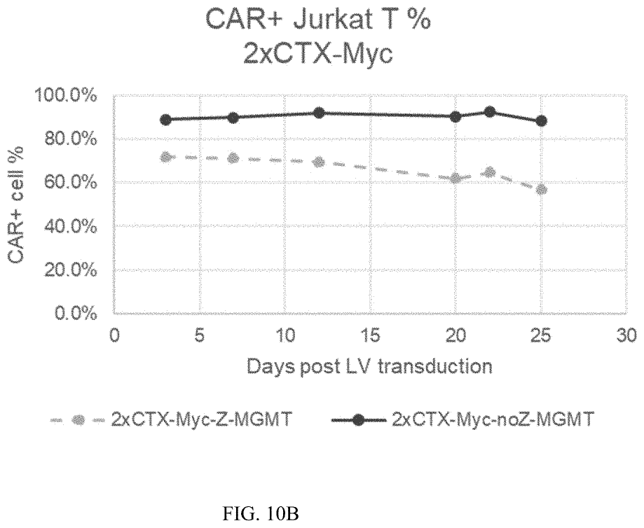

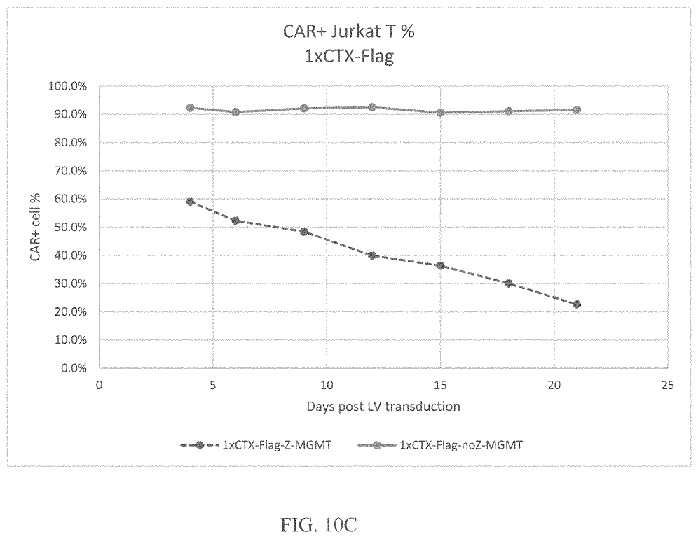

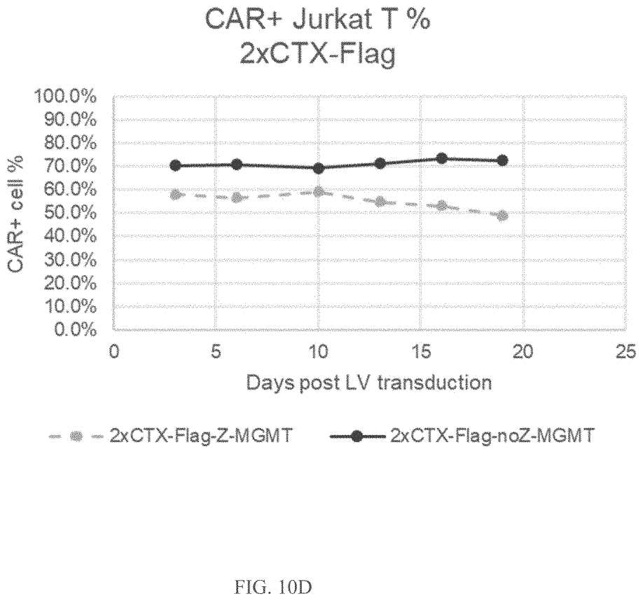

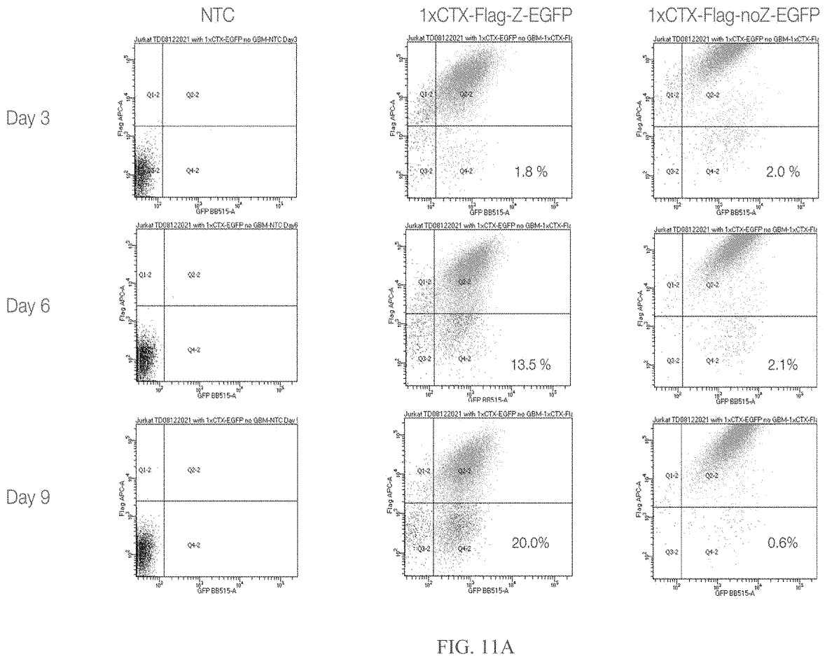

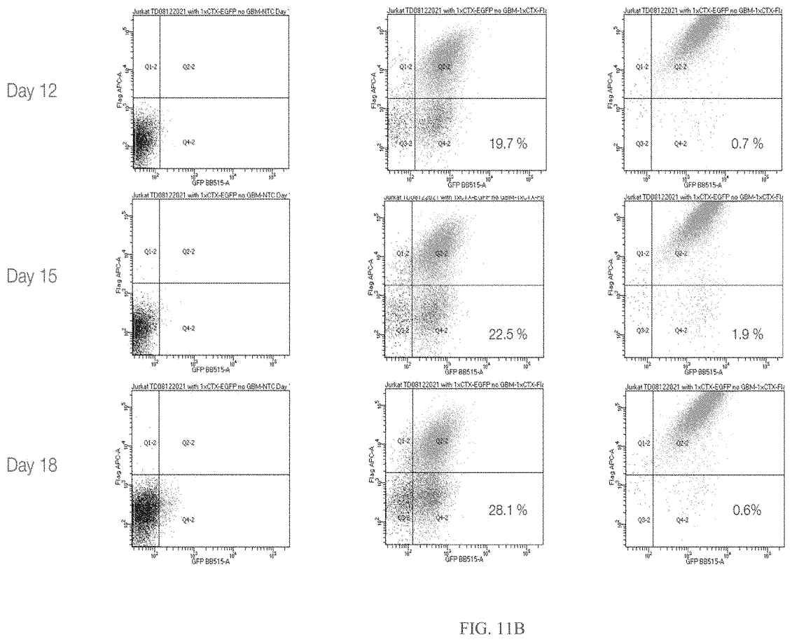

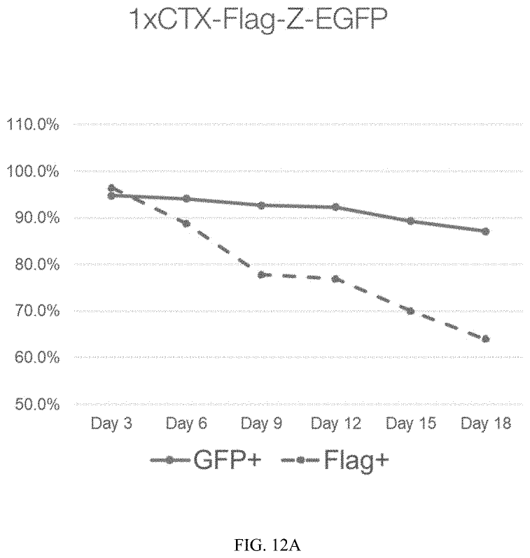

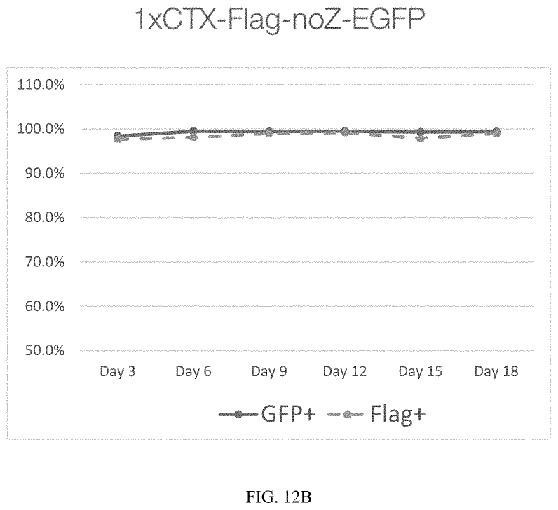

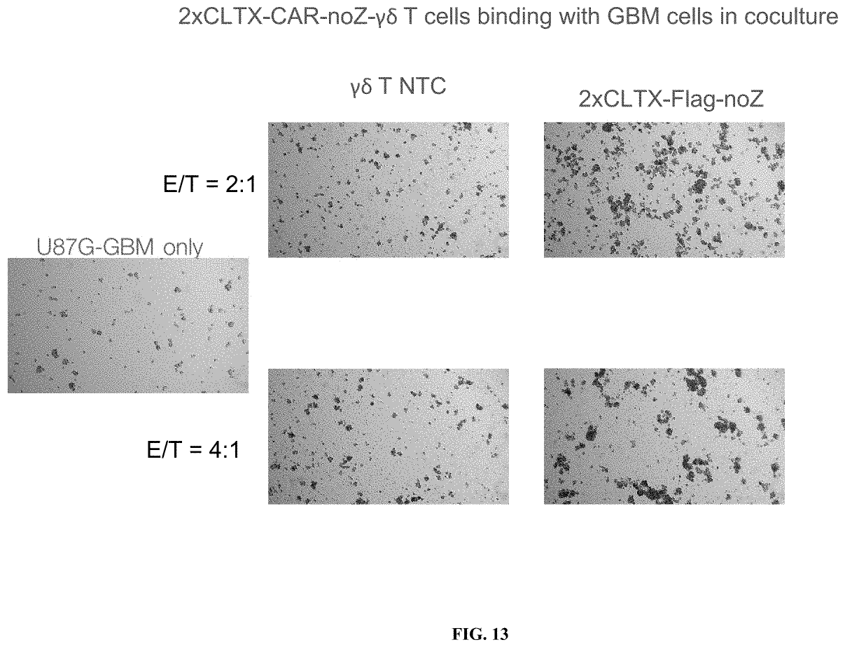

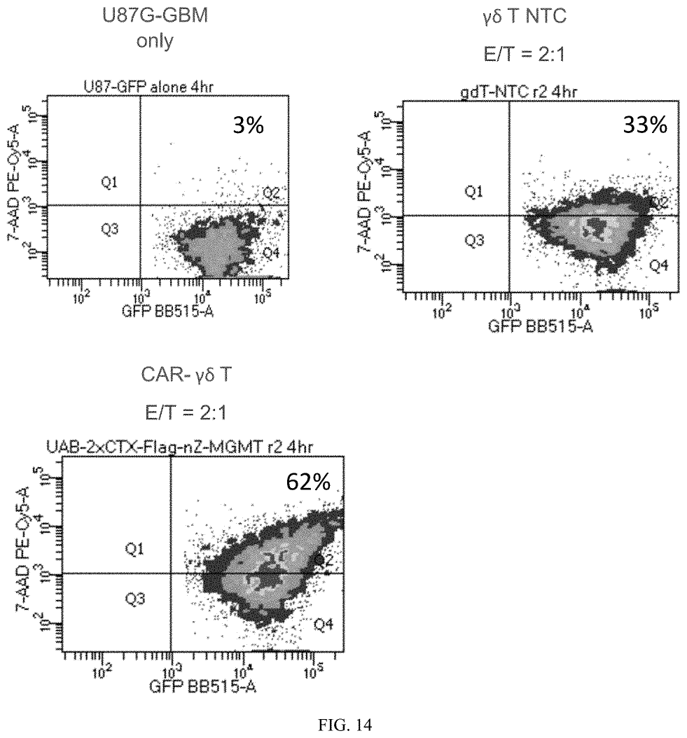

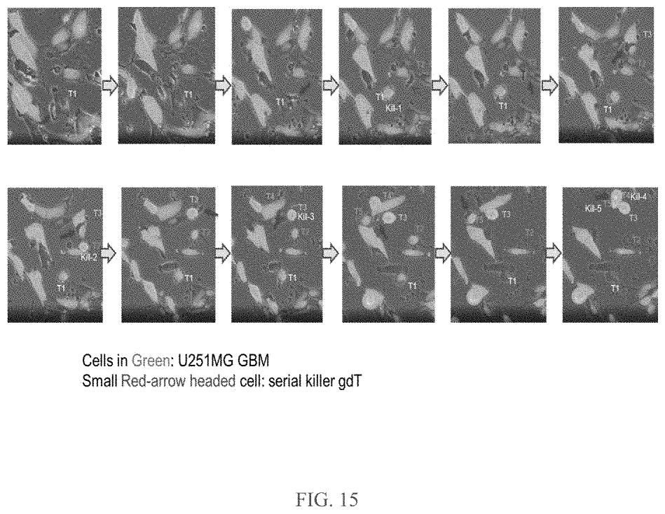

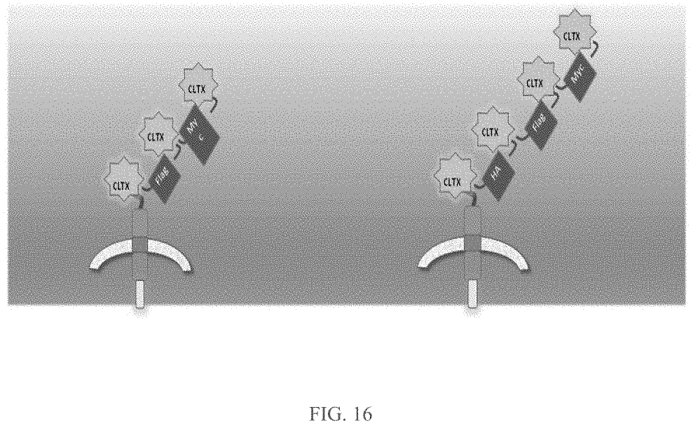

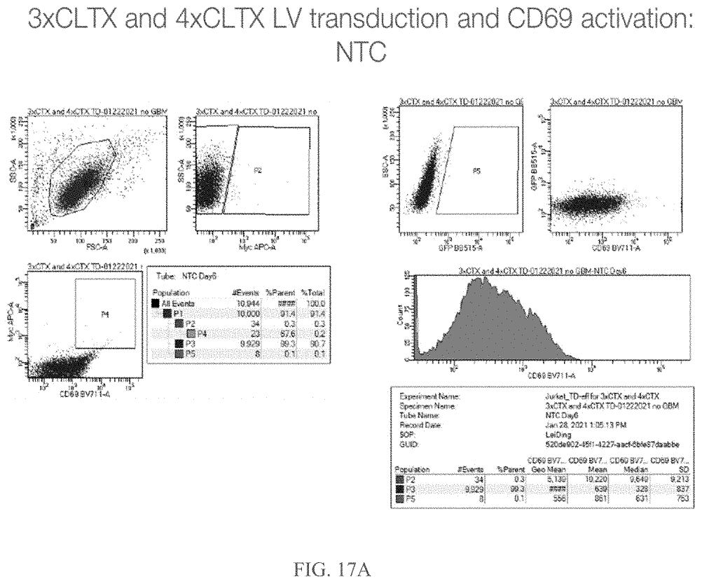

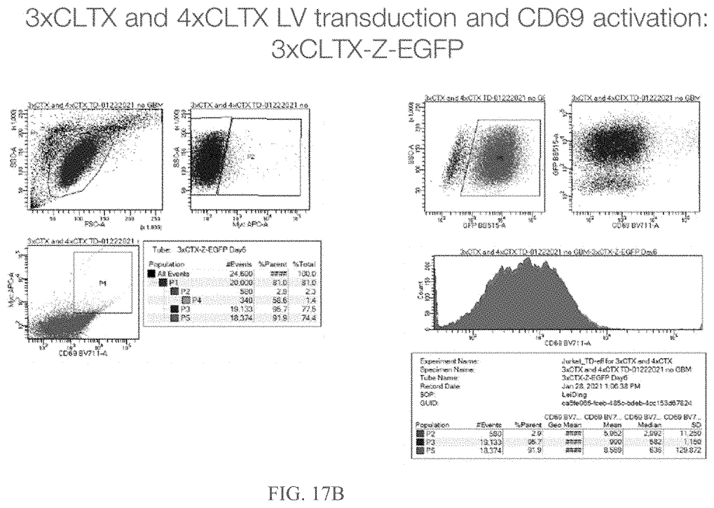

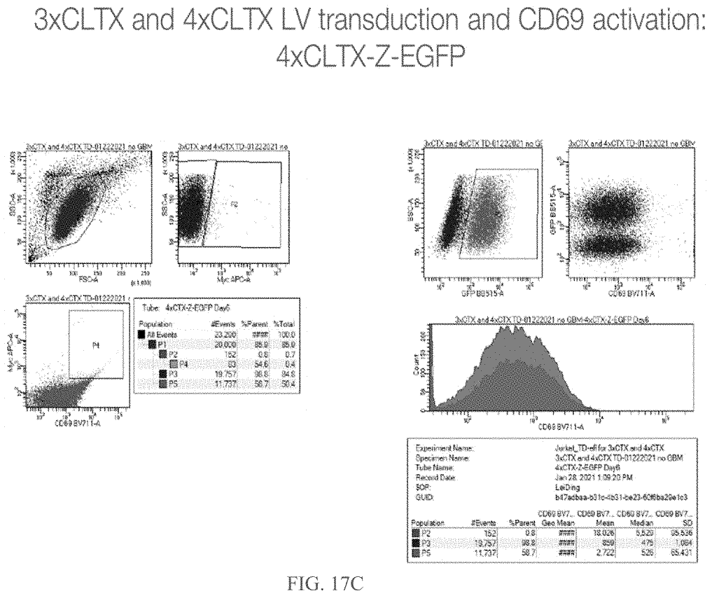

a. CLTX-Myc-CLTX-CD8H-CD28co-Z-EGFP;

b. CLTX-Myc-CLTX-CD8H-CD28co-noZ-EGFP;

c. CLTX-Myc-CLTX-CD8H-CD28co-Z-MGMT;

d. CLTX-Myc-CLTX-CD8H-CD28co-noZ-MGMT;

e. CLTX-Flag-CLTX-CD8H-CD28co-Z-EGFP;

f. CLTX-Flag-CLTX-CD8H-CD28co-noZ-EGFP;

g. CLTX-Flag-CLTX-CD8H-CD28co-Z-MGMT; and

h. CLTX-Flag-CLTX-CD8H-CD28co-noZ-MGMT.

The depicted CLTX-CAR constructs also include the following sCLTX (single CTLX peptide) constructs:

a. CLTX-Myc-CD8H-CD28co-Z-EGFP;

b. CLTX-Myc-CD8H-CD28co-noZ-EGFP;

c. CLTX-Myc-CD8H-CD28co-Z-MGMT;

d. CLTX-Myc-CD8H-CD28co-noZ-MGMT;

e. CLTX-Flag-CD8H-CD28co-Z-EGFP;

f. CLTX-Flag-CD8H-CD28co-noZ-EGFP;

g. CLTX-Flag-CD8H-CD28co-Z-MGMT; and

h. CLTX-Flag-CD8H-CD28co-noZ-MGMT.

FIG. 3 shows flow cytometric analysis of cell surface staining using anti-Myc monoclonal antibody and shows CLTX cell surface localization of the dCLTX-CAR. The cells are also gated for GFP showing co-expression of dCLTX-CAR and the marker gene.

FIG. 4 is a bar graph showing that dCLTX-CARs Jurkat T-cells expressing EGFP (“CMC-EGFP”) and MGMT (“CMC-MGMT”) displayed enhanced CD69 activation as compared to the sCLTX-CAR Jurkat T-cell expressing EGFP (“CTX-EGFP”). The dCLTX-CAR cells included a Flag peptide between the two CTLX peptides.

FIG. 5 shows flow cytometric analysis of lentivirus-transduced Jurkat T cells that were co-cultured with U251 GBM cells for 24 hours and stained with anti-CD69 antibody.

FIG. 6 shows graphs of percentage CLTX-CAR-transduced Jurkat cells (left: 1×CLTX-Myc and right: 2×CLTX-Myc) over time (days).

FIG. 7 shows photographs of GBM cells co-cultured with γδ T cells. The top left panel shows U25-GFP cells only. GBM cells treated with 1×CLTX-Flag-noZ and 2×CLTX-Flag-noZ are shown at the bottom left and bottom right, respectively.

FIG. 8 shows flow cytometric analysis of transduced γδ T cells co-cultured with U251-GFP or U87-GFP cells at different ratios for 24-48 hours followed by staining with Annexin V and 7-AAD.

FIG. 9 shows flow cytometric analysis of CLTX-CAR transduced Jurkat T cells that were co-cultured with U251 GBM cells for 24 hours and stained with anti-CD69 antibody. Data is shown for control cells (NTC), green fluorescence protein control (GFP), 1×CLTX-Z-GFP (a single CLTX peptide, CD3z signaling domain and GFP without a tag), 1×CLTX-noZ-GFP (a single CLTX peptide, no CD3z signaling domain and GFP without a tag), 1×CLTX-Flag-Z (a single CLTX peptide, a Flag peptide and a CD3z signaling domain), 1×CLTX-Flag-NoZ (a single CLTX peptide, a Flag peptide, and no CD3z signaling domain), 2×CLTX-Flag-Z (two CLTX peptides, a Flag peptide and a CD3z signaling domain) and 2×CLTX-Flag-noZ (two CLTX peptides, a Flag peptide and no CD3z signaling domain). FIG. 9 (top panel) show schematics depicting 1×CLTX constructs with a Flag or Myc peptide and 2×CLTX constructs with a Flag or Myc peptide linking the two CLTX peptides. As shown in in the figure, Jurkat T cells transduced with CLTX-CAR constructs with no CD3z signaling domains (noZ) showed no CD69 activation upon U251 co-culture. Jurkat T cells transduced with 1×CLTX-CAR with no tag showed moderate activation of CD69 compared to non-transduced cells. Jurkat T cells transduced with 1×CLTX-CAR or 2×CLTX-CARs with a Flag tag showed more greatly elevated CD69 activation.

FIGS. 10A-10D are graphs of percentage CLTX-CAR-transduced Jurkat cells over time (days). FIG. 10A is a graph showing percentage of 1×CTX-Myc-Z-MGMT cells (one CLTX peptide, Myc peptide, CD3z signaling domain, and MGMT) and 1×CTX-Myc-noZ-MGMT cells (one CLTX peptide, Myc peptide, no CD3z signaling domain, and MGMT) over time (days). FIG. 10B is a graph showing percentage of 2×CTX-Myc-Z-MGMT cells (two CLTX peptides, Myc peptide, CD3z signaling domain, and MGMT) and 2×CTX-Myc-noZ-MGMT cells (two CLTX peptide, Myc peptide, no CD3z signaling domain, and MGMT) over time (days). FIG. 10C is a graph showing percentage of 1×CTX-Flag-Z-MGMT cells (one CLTX peptide, Flag peptide, CD3z signaling domain, and MGMT) and 1×CTX-Flag-noZ-MGMT cells (one CLTX peptide, Flag peptide, no CD3z signaling domain, and MGMT) over time (days). FIG. 10D is a graph showing percentage of 2×CTX-Flag-Z-MGMT cells (two CLTX peptides, Flag peptide, CD3z signaling domain, and MGMT) and 2×CTX-Flag-noZ-MGMT cells (two CLTX peptides, Flag peptide, no CD3z signaling domain, and MGMT) over time (days). As shown in FIGS. 10A-10D, cells with no signaling domain had greater persistence than cells with the CD3z signaling domain and cells with two CLTX peptides (dual CLTX) with a CD3z signaling domain showed greater persistence than comparable cells with only one CLTX peptide.

FIGS. 11A and 11B show flow cytometric analysis of CLTX-CAR transduced Jurkat cells expressing enhanced green fluorescent protein (EGFP) as an internal reporter at Days 3, 6, 9, 12, 15, and 18. The data shown is for control cells (NTC), 1×CTX-Flag-Z-EGFP (one CLTX peptide, Flag peptide, CD3z signaling domain and EGFP) and 1×CTX-Flag-noZ-EGFP (one CLTX peptide, Flag peptide, no signaling domain and EGFP). 1×CTX-Flag-noZ-EGFP showed greater persistence than 1×CTX-Flag-Z-EGFP cells. Specifically, the 1×CLTX-Flag-Z-EGFP transduced Jurkat T cells showed declined percentage of cells expressing CLTX-CAR on the cell surface over time while the EGFP is still present intracellularly in those cells.

FIGS. 12A and 12B are graphs of percentage 1×CLTX-CAR-transduced Jurkat cells over time (days) from the flow cytometric analysis shown FIGS. 11A and 11B. FIG. 12A is a graph showing percentage of 1×CTX-Flag-Z-EGFP cells and cells transduced with GFP alone (control) over time (days). FIG. 12B is a graph showing 1×CTX-Flag-noZ-EGFP and cells transduced with GFP over time (days).

FIG. 13 shows photographs of control γδ T cells (γδ T cells NTC) and 2×CLTX-CAR-noZ-γδ T cells binding to GBM cells in co-culture at effector/target (E/T) of 2:1 and E/T of 4:1. The left-most panel shows U87G-GBM cells only. 2×CLTX-CAR-FLAG-noZ-γδ T cells showed greater binding of GBM cells than control γδ T cells.

FIG. 14 shows flow cytometric analysis of control (γδ T NTC), 2×CLTX-CAR-noZ-γδ T (CAR-γδ T) cells that were co-cultured with U87-GFP glioblastoma cells at E/T of 2.1 for 4 hours followed by staining with 7-AAD. The cells are sorted for GFP and 7-AAD. The U87GFP GBM cells are GFP+ and the dead cells are 7-AAD+. The left-most panel shows U87G-GBM cells alone.

FIG. 15 shows that 2×CLTX-CAR-noZ-γδ T showed enhanced cytotoxicity to U87 glioblastoma cells as compared to control γδ T cells (without the CAR) even without a CD3z signaling domain.

FIG. 15 show a series of still images from a time lapse movie showing serial killing of U251MG GBM cells by 2×CLTX-CAR-noZ γδ T cells. The green cells (or as shown in gray-scale, the brighter cells) in the images are the tumor cells. The red arrows indicate the γδ T cell over time as it binds and kills different tumor cells (see T1, T2, T3, T4 and T5 and Kill-1, Kill-2, Kill-3, Kill-4 and Kill-5 in the images). The images were taken over time (left to right, and as indicated by the arrows between the images).

FIG. 16 is a schematic showing a 3×CLTX-CAR and 4×CLTX-CAR in a cell. The three CLTX peptides in the 3×CLTX-CAR are linked with a Flag peptide and a Myc peptide as shown. The four CLTX peptides in the 4×CLTX-CAR are linked with a HA, a Flag peptide and a Myc peptide as shown. Also shown are the hinge region, the transmembrane region, the optional intracellular co-stimulatory domain, and the optional intracellular signaling domain. For 3×CLTX-CAR constructs, the constructs include:

a. CTX-Myc-CTX-Flag-CTX-CD8H-CD28co-Z-EGFP;

b. CTX-Myc-CTX-Flag-CTX-CD8H-CD28co-noZ-EGFP;

c. CTX-Myc-CTX-Flag-CTX-CD8H-CD28co-Z-MGMT; and

d. CTX-Myc-CTX-Flag-CTX-CD8H-CD28co-noZ-MGMT

For 4×CLTX-CAR constructs, the constructs include:

a. CTX-Myc-CTX-Flag-CTX-HA-CTX-CD8H-CD28co-Z-EGFP;

b. CTX-Myc-CTX-Flag-CTX-HA-CTX-CD8H-CD28co-noZ-EGFP;

c. CTX-Myc-CTX-Flag-CTX-HA-CTX-CD8H-CD28co-Z-MGMT; and

d. CTX-Myc-CTX-Flag-CTX-HA-CTX-CD8H-CD28co-noZ-MGMT.

FIGS. 17A-17C show flow cytometric analysis of cell surface staining using anti-Myc monoclonal antibody for control (NTC) cells, 3×CLTX-Z-EGFP Jurkat cells and 4×CLTX-Z-EGFP Jurkat cells and also shows CD69 activation after co-culturing with U251 GBM cells for 24 hours and staining with anti-CD69 antibody. The cells are also gated for GFP. FIGS. 17B and 17C show that the 3×CLTX and 4×CLTX CARs are not presented normally on the cell surface (GFP+ is measured but not Myc). In addition, no CD69 activation was observed in 3×CLTX or 4×CLTX transduced Jurkat cells after co-cultured with tumor cells.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

As used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt. % to about 5 wt. %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”. Numbers, ratios, concentrations, amounts, ranges and other numerical data should be construed as modified by the term “about” unless inconsistent with the context.

The term “drug resistant immunotherapy” or DRI is a strategy for treating cancer whereby anti-cancer immune cells, preferably γδ T-cells, are genetically engineered to resist the toxic effects of chemotherapy drugs which allows for the combined administration of chemotherapy and immunotherapy. Chemotherapy resistance or the acquisition of chemoresistance is a well-known phenomenon in the field of cancer treatment. Such resistance to chemotherapeutic agents can arise from the expression of certain DNA, RNA or polypeptides that impact drug resistance genes, expression of a gene that conveys drug resistance, the expression of a polypeptide that confers resistance to chemotherapeutic agents. The DRI strategy described herein uses chemoresistance to confer resistance to the immune cells that can be used in cancer immunotherapy. DRI γδ T-cells, for example, include γδ T-cells that have been genetically engineered to express a survival factor as described herein, including, but not limited to, a DNA, RNA or polypeptide that confers resistance to a chemotherapeutic agent. A polypeptide that confers resistance to a chemotherapeutic agent can be referred to herein a as a “survival polypeptide”). DRI γδ T-cells that comprise the multivalent CLTX-CAR can be referred to as multivalent CLTX-CAR DRI γδ T-cells.

The term “survival factor” refers to any agent now known or later discovered in the art that confers resistance to a chemotherapeutic agent, and/or to a chemotherapeutic agent treatment regimen and/or allows the cells comprising the survival factor to survive in a treatment environment (such as a chemotherapy treatment environment). The phrase “confers resistance” and the like encompasses the acquisition of resistance to a chemotherapeutic agent or improvement in resistance to a chemotherapeutic agent. The “survival factor” includes an agent that confers resistance to a chemotherapeutic agent when it is expressed by the γδ T cell. The “survival factor” can thus be a DNA, RNA or polypeptide that is expressed by the γδ T cells (e.g., encoded by a drug resistance gene) and that confers resistance to a chemotherapeutic agent. As described herein, the γδ T cell can be engineered to express the DNA, RNA or polypeptide that confers resistance to a chemotherapeutic drug by including a vector which expresses a gene, a gene fragment, a DNA, an siRNA, or an mRNA, that encodes the survival factor that confers resistance to a chemotherapeutic agent. In yet other aspects, the survival factor is a DNA that confers resistance to a chemotherapeutic agent. In further aspects, the survival factor is an RNA (e.g., a RNAi, siRNA, microRNA, or mRNA) confers resistance to a chemotherapeutic agent.

In certain aspects, the survival factor is a polypeptide that confers resistance to a chemotherapeutic agent; for example, the polypeptide confers resistance when it is expressed by the γδ T cells. In certain embodiments, the survival factor is MGMT, multidrug resistance protein 1 (MDR1), or 5′ nucleotidase II (NT5C2). Other survival factors include, for example, a drug resistant variant of dihydrofolate reductase (L22Y-DHFR) and thymidylate synthase. In certain aspects, the survival factor in is MGMT. Other polypeptides that confer resistance may be used or expressed by the cell depending on the nature of the treatment environment (i.e., what other treatment regimens are being given to the patient in combination with the cells compositions of the present disclosure). MGMT repairs alkylating lesions of the DNA by removing mutagenic adducts from the O6 position of guanine. Such mutagenic adducts can be caused by alkylating agents (including, but not limited to, temozolomide). Thus, MGMT is a polypeptide that confers resistance to alkylating agents such as temozolomide. The survival factor can be a polypeptide that confers resistance to a chemotherapeutic agent, including, but not limited, the specific chemotherapeutic agents described herein.

By “administration” is meant introducing a compound, biological materials including a cell population, or a combination thereof, or a composition comprising any of the aforementioned compounds, biological materials (e.g., a cell population), or a combination thereof, of the present invention into a human or animal subject. One preferred route of administration of the compounds is intravenous. Another preferred route is parenteral. “Parenteral” refers to a route of administration that is associated with injection, including intraorbital, infusion, intraarterial, intracapsular, intracardiac, intradermal, intramuscular, intraperitoneal, intrapulmonary, intraspinal, intrasternal, intrathecal, intracranial, intrauterine, intravenous, subarachnoid, subcapsular, subcutaneous, transmucosal, or transtracheal. Other exemplary routes of administration of the compounds may be intraperitoneal or intrapleural, or via a catheter to the brain. However, any route of administration, such as oral, topical, subcutaneous, peritoneal, intra-arterial, inhalation, vaginal, rectal, nasal, introduction into the cerebrospinal fluid, intracranial, or instillation into body compartments can be used. Direct injection into a target tissue site such as a solid tumor is also contemplated. For example, intracranial administration of the γδ T-cells for the treatment of a glioma or other intracranial tumor can be used.

The term “cancer”, as used herein, shall be given its ordinary meaning, as a general term for diseases in which abnormal cells divide without control. Cancer cells can invade nearby tissues and can spread through the bloodstream and lymphatic system to other parts of the body. When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. Carcinoma is cancer that begins in the skin or in tissues that line or cover internal organs. Glioma is a tumor that arises from the supportive (“gluey”) tissue of the brain, called glia, which helps to keep the neurons in place and functioning well. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

Representative cancers include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Glioblastoma, Childhood; Glioblastoma, Adult; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet-cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chrome Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ. Cell Tumor, Childhood; Extragonadal Germ. Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hvpopharyngeal Cancer; Hypothalamic and Visual Pathway Glioma, Childhood; Intraocular Melanoma; Islet-cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chrome Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chrome; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet-cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a different environment, not normally conducive to their growth), and continue their rapid growth and division in this new location. This is called metastasis. Once malignant-cells have metastasized, achieving a cure or treatment is more difficult. Benign tumors have less of a tendency to invade and are less likely to metastasize.

The term “fusion protein”, as used herein, refers to chimeric molecules, which comprise, for example, an antigen recognition domain for example, comprising at least two CLTX peptides, and at least one heterologous portion, i,e, a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship. The multivalent CLTX-CARs described herein can be fusion proteins comprising at least two CLTX peptides.

The methods of treatment described herein comprising administration of the multivalent CLTX-CAR γδ T-cells and a chemotherapeutic agent can be used to reduce a cancer or tumor. The terms “reducing a cancer,” “inhibition of cancer,” “inhibiting cancer,” “preventing cancer recurrence,” and similar terms and are used interchangeably herein and refer to one or more of a reduction in the size or volume of a tumor mass, a decrease in the number of metastasized tumors in a subject, a decrease in the proliferative status (the degree to which the cancer cells are multiplying) of the cancer cells, prevention of recurrences of previous tumors or the development of new metastases, and the like.

The method of treatment described herein comprising administration of the multivalent CLTX-CAR γδ T-cells and a chemotherapeutic agent can be used to reduce a tumor. The term “reducing a tumor” as used herein refers to a reduction in the size or volume of a tumor mass, a decrease in the number of metastasized tumors in a subject, a decrease in the proliferative status (the degree to which the cancer cells are multiplying) of the cancer cells, and the like.

The term “chemotherapeutic agent” as used herein refers to a compound or a derivative thereof that can interact with a cancer cell, thereby reducing the proliferative status of the cell and/or killing the cell for example, by impairing cell division or DNA synthesis, or by damaging DNA, effectively targeting fast dividing cells. Examples of chemotherapeutic agents include, but are not limited to, alkylating agents (e.g., cyclophosphamide, ifosfamide, temozolomide); metabolic antagonists (e.g., methotrexate (MTX), 5-fluorouracil or derivatives thereof); a substituted nucleotide; a substituted nucleoside; DNA demethylating agents (also known as antimetabolites; e.g., azacitidine); antitumor antibiotics (e.g., mitomycin, adriamycin); plant-derived antitumor agents (e.g., vincristine, vindesine, TAXOL®, paclitaxel, abraxane); cisplatin; carboplatin; etoposide; and the like. Such agents may further include, but are not limited to, the anti-cancer agents trimethotrexate (TMTX); temozolomide (TMZ); raltitrexed; S-(4-Nitrobenzyl)-6-thioinosine (NBMPR); 6-benzyguanidine (6-BG); a nitrosoureas a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, and Streptozocin (Streptozotocin)); cytarabine; and camptothecin; or a therapeutic derivative of any thereof.

The term “chimeric antigen receptor(s) (CAR(s)),” as used herein, refers to artificial T-cell receptors, T-bodies, single-chain immunoreceptors, chimeric T-cell receptors, or chimeric immunoreceptors, for example, and encompass engineered receptors that graft an artificial specificity (for example, an antigen recognition domain) onto a particular immune effector cell, for example, γδ T-cells. In some embodiments, CARs comprise an intracellular activation domain, a transmembrane domain, and an extracellular domain that may vary in length and that comprises an antigen recognition domain. Also as described herein, a CAR can lack an intracellular signaling domain. In multivalent CLTX-CARs, the antigen recognition domain can comprise more than one CLTX peptide, for example, two, three, four, five, or more CLTX peptides. A CAR comprising a CLTX peptide within its antigen recognition domain is referred to herein as a CLTX-CAR. A “multivalent CLTX-CAR” is a CTLX-CAR that comprises more than one CLTX peptides in the antigen recognition domain; for example, the more than one CLTX peptides can be linked by a peptide or peptides. Such linking peptides are referred to herein as the “linker peptide” or the “linking peptide.” The linker peptide that links a pair of CLTX peptides can be the same or different from the peptide that links a different pair of CLTX peptides in the antigen recognition domain; for example, in a multivalent CLTX-CAR comprising three CLTX peptides, the linker peptide that links two peptides (e.g., the first and the second peptide) can be the same or different from the linker peptide that links (e.g. the second peptide and the third peptide). The term “dual CLTX-CAR” or “dCLTX-CAR” or “divalent CAR” or “2×CLTX-CAR” refers to a CAR comprising two CLTX peptides in the antigen recognition domain; the two CLTX peptides can be attached by a linker peptide. A “single CLTX-CAR” or “sCLTX-CAR” or “1×CLTX-CAR” refers to a CAR comprising only one CLTX peptides in the antigen recognition domain.

As used herein, the terms “chlorotoxin” and “CLTX” or “CTX” are used interchangeably and refer to a scorpion venom peptide, chlorotoxin, that comprises 36 amino acids having the amino acid sequence: MCMPCFTTDHQMARKCDDCCGGKGRGKCYGPQCLCR (SEQ ID NO: 1) (UniProt Accession #P45639). Without wishing to bound by theory, the CLTX peptide binding domain can act to enhance trafficking of the γδ T cells to solid tumors including, but not limited to, gliomas, liver cancer, ovarian cancers and others that express the target. The CLTX peptide can also enhance trafficking to melanoma, small cell lung carcinoma, neuroblastoma, breast cancer, kidney cancer, liver cancer, lung cancer, ovarian cancer, and medulloblastoma.

The specificity of other CAR designs may be derived from ligands of receptors (e.g., peptides). In certain cases, the spacing of the antigen-recognition domain can be modified to reduce activation-induced cell death. In certain cases, the intracellular signaling domain of the CARs comprise domains for additional co-stimulatory signaling, such as, but not limited to, FcR, CD27, CD28, CD 137, DAP 10, and/or OX40 in addition to CD3ζ. In some cases, molecules can be co-expressed with the CAR, including co-stimulatory molecules, reporter genes for imaging (e.g., for positron emission tomography), gene products that allow host-cells expressing the CAR to survive in a treatment environment created by an additional therapeutic treatment, gene products that conditionally ablate the host-cells expressing the CAR upon addition of a pro-drug, homing receptors, chemokines, chemokine receptors, cytokines, and cytokine receptors.

Also as described herein, a CLTX-CAR can be a CLTX-CAR that does not comprise an intracellular signaling domain.

The invention also encompasses multivalent CARs that comprises more than one functional variant of a CLTX peptide, wherein the functional variants can be the same or different. The invention further includes multivalent CARs that comprises more than one CLTX-like peptide, wherein the CLTX-like peptides can be the same or different. The invention additionally encompasses a multivalent CAR that comprises more than one (for example, two) of the following: a CLTX peptide, a CLTX-like polypeptide, and a functional variants of CLTX peptide in the extracellular antigen binding domain. For example, a divalent CAR can comprise two CLTX peptides, or two CLTX-like polypeptides, or two functional variants of a CLTX peptide, or a combination thereof (e.g., one CLTX peptide and one CLTX-like polypeptide).

A “functional variant” of a CLTX peptide is a peptide having substantial or significant sequence identity or similarity to chlorotoxin (CLTX) (e.g., SEQ ID NO: 1), wherein the functional variant retains the biological activity of the chlorotoxin peptide. For example, a CAR comprising a functional variants of CLTX retains at least some of the biological activity of a CLTX-CAR; for example, retains the ability to recognize target-cells to a similar extent, the same extent, or to a higher extent, as the parent CLTX-CAR. The terms “functional variant of CLTX” and “functional variant of a CLTX peptide” are used interchangeably herein. A functional variant of a CLTX peptide can be, or can have an amino acid sequence that is, at least about 65% identical, at least about 80% identical, about 90% identical, about 95% identical, or about 99% identical to the CLTX peptide (e.g., SEQ ID NO: 1). For example, the multivalent CAR as described herein can utilize at least one functional variant of CLTX within the extracellular antigen recognition domain (the antigen recognition moiety can further comprise a CLTX peptide, another functional of CLTX, or a CLTX-like peptide), wherein such functional variant of CLTX comprises a sequence which has 70%, 80%, 90%, 95% or greater homology with SEQ ID NO: 1.

A functional variant can, for example, comprise the amino acid sequence of CLTX with at least one amino acid modification (such, as but not limited to, deletions, insertions and substitutions) can be selected, as would be known to one of ordinary skill in the art, to generate a desired CTX-CAR functional variant. Conservative modifications to the amino acid sequence of SEQ ID NO: 1 (and the corresponding modifications to the encoding nucleotides) will produce functional variants having functional and chemical characteristics similar to those of naturally occurring CLTX.

The phrase “conservative amino acid substitution” or “conservative mutation” refers to the replacement of one amino acid by another amino acid with a common property. Examples of conservative mutations include amino acid substitutions of amino acids within the same amino acid subgroup, for example, lysine for arginine and vice versa such that a positive charge may be maintained; glutamic acid for aspartic acid and vice versa such that a negative charge may be maintained; serine for threonine such that a free —OH can be maintained; and glutamine for asparagine such that a free —NH₂ can be maintained. A “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine. Conservative amino acid substitutions also encompass non-naturally occurring amino acid residues which are typically incorporated by chemical peptide synthesis rather than by synthesis in biological systems. These include peptidomimetics, and other reversed or inverted forms of ammo acid moieties. It will be appreciated by those of skill in the art that nucleic acid and polypeptide molecules described herein may be chemically synthesized as well as produced by recombinant means. Naturally occurring residues may be divided into classes based on common side chain properties: 1) hydrophobic: norleucine, Met, Ala, Val, Leu, lie; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Tip, Tyr, Phe. Non-conservative amino acid substitutions are also contemplated, particularly when such non-conservative amino acids occur in related polypeptides with similar activity. For example, non-conservative substitutions may involve the exchange of a member of one of the amino acid classes for a member from another class. Such substituted residues may be introduced into regions of the CLTX or CLTX-like peptide functional variants that are homologous with related CLTX polypeptide orthologs, or into the non-homologous regions of the molecule. In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is understood in the art (Kyle et al., J. Mol. Biol., 157: 105-131, 1982). It is known that certain amino acids may be substituted for other amino acids having a similar hydropathic index or score and still retain a similar biological activity. In making changes based upon the hydropathic index, the substitution of amino acids whose hydropathic indices are within +/−2 may be used; in an alternate embodiment, the hydropathic indices are with +/−1; in yet another alternate embodiment, the hydropathic indices are within +/−0.5. It is also understood in the art that the substitution of like amino acids can be made effectively on the basis of hydrophilicity. The greatest local average hydrophilicity of a polypeptide as governed by the hydrophilicity of its adjacent amino acids, correlates with a biological property of the protein. The following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5 1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within +/−2 may be used; in an alternate embodiment, the hydrophilicity values are with +/−1; in yet another alternate embodiment, the hydrophilicity values are within +/−0.5.

Desired amino acid substitutions (whether conservative or non-conservative) can be determined by those skilled in the art at the time such substitutions are desired. For example, amino acid substitutions can be used to identify important residues of a CLTX or to increase or decrease the affinity of a CLTX with a particular binding target in order to increase or decrease an activity (for example, an effector function and/or an immune effector function) of a CLTX-CAR of the present disclosure.

In one embodiment, a functional variant of CLTX is one that contains one or more substitutions at positions corresponding to positions 1, 3, 10, 13, 14, 17, 25 and 36 (positions with reference to SEQ ID NO: 1). In one aspect of such an embodiment, preferable substitutions for such CLTX functional variants at the indicated positions include: Arg for Met at position 1; Lys or Ser for Met at position 3; Pro or Gln for His at position 10; Ser or Thr for Ala at position 13; Lys for Arg at position 14; Ala or Tyr for Asp at position 17; Lys for Arg at position 25; and Ala for Arg at position 36. In certain aspects of such embodiments, the functional variant of CTX contains 6 or fewer substitutions from the indicated positions, 4 or fewer substitutions from the indicated positions or 2 or fewer substitutions from the indicated positions.

In one embodiment, a functional variant of CLTX is one that contains one or more substitutions at positions corresponding to positions 9-11, 14-15, 17-18, 25 and 29, with or without a deletion of amino acids at positions 23 and 24 (positions with reference to SEQ ID NO: 1). In one aspect of such embodiment, preferable substitutions for such CTX functional variants at the indicated positions include: Arg for Asp at position 9; Pro or Gln for His at position 10; Asn or Asp for Gln at position 11; Lys, Gln or Asn for Arg at position 14; Arg or Gln for Lys at position 15; Asn, Ala, Arg or Tyr for Asp at position 17; Glu or Ala for Asp at position 18; Tyr, Lys, He, Gly or Asn for Arg at position 25; Phe for Tyr at position 29; and Asn or Ala for Arg at position 36. In another aspect of such embodiment, preferable substitutions for such CTX functional variants at the indicated positions include: Arg for Asp at position 9; Pro for His at position 10; Asn for Gln at position 11; Lys or Gln for Arg at position 14; Gln for Lys at position 15; Arg for Asp at position 17; Ala for Asp at position 18; Asn for Arg at position 25; Phe for Tyr at position 29; and Asn for Arg at position 36. In certain aspects of such embodiments, the functional variant of CTX contains 6 or fewer substitutions from the indicated positions, 4 or fewer substitutions from the indicated positions or 2 or fewer substitutions from the indicated positions.

In one embodiment, a functional variant of CLTX is one that contains substitutions at positions corresponding to positions 1, 3, 9-15, 17-18, 21, 25-26, 29-31 and 36 with or without a deletion of amino acids at positions 23 and 24 (positions with reference to SEQ ID NO: 1). In one aspect of such embodiment, preferable substitutions for such CTX functional variants at the indicated positions include: Arg for Met at position 1; Lys, Ser or Gly for Met at position 3; Arg for Asp at position 9; Pro or Gln for His at position 10; Asn or Asp for Gln at position 11; Tyr for Met at position 12; Ser, Thr or Glu for Ala at position 13; Lys, Gln or Asn for Arg at position 14; Arg or Gln for Lys at position 15; Asn, Ala, Arg or Tyr for Asp at position 17; Glu or Ala for Asp at position 18; Arg or Lys for Gly at position 21; Tyr, Lys, Ile, Gly or Asn for Arg at position 25; Lys for Gly at position 27; Phe for Tyr at position 29; Phe for Gly at position 30; Gly or Tyr for Asp at position 31; and Asn or Ala for Arg at position 36. In certain aspects of such embodiments, the functional variant of CLTX contains 12 or fewer substitutions from the indicated positions, 10 or fewer substitutions from the indicated positions, 8 or fewer substitutions from the indicated positions, 6 or fewer substitutions from the indicated positions, 4 or fewer substitutions from the indicated positions or 2 or fewer substitutions from the indicated positions.

In another embodiment, the functional variant of CLTX is a polypeptide having the sequence of amino acids 2-36 of SEQ ID NO: 1. In one aspect of such embodiment, the CLTX variant may have the amino acid substitutions described for amino acids 1, 3, 10, 13, 14, 17, 25 and 36, the ammo acid substitutions described for amino acids 9-11, 14-15, 17-18, 25 and 29 above or the amino acid substitutions described for amino acids 1, 3, 9-15, 17-18, 21, 25-26, 29-31 and 36 above.

In another embodiment, the functional variant of CLTX is a polypeptide having the sequence of amino acids 1-35 of SEQ ID NO: 1. In one aspect of such embodiment, the CLTX variant may have the amino acid substitutions described for amino acids 1, 3, 10, 13, 14, 17, 25 and 36, the ammo acid substitutions described for amino acids 9-11, 14-15, 17-18, 25 and 29 above or the amino acid substitutions described for amino acids 1, 3, 9-15, 17-18, 21, 25-26, 29-31 and 36 above. In another embodiment, the functional variant of CLTX is a polypeptide having the sequence of amino acids 2-35 of SEQ ID NO: 1. In one aspect of such embodiment, the CLTX variant may have the amino acid substitutions described for amino acids 1, 3, 10, 13, 14, 17, 25 and 36, the ammo acid substitutions described for amino adds 9-11, 14-15, 17-18, 25 and 29 above or the amino acid substitutions described for amino acids 1, 3, 9-15, 17-18, 21, 25-26, 29-31 and 36 above.

A “chlorotoxin-like peptide” is a peptide that has a similar primary structure to that of CLTX and includes those described, for example, in WO2018107134, the contents of which are expressly incorporated by reference herein. Such peptides include, but are not limited to, Bs8 (Uniprot Acc. No. PI 5229), Insectotoxin-I4 (UniProt Acc No P60269), Lqh 8/6 (UniProt Acc No. P55966), Insectotoxin-13 (UniProt Acc No P60268), Insectotoxin-I5A (UniProt Acc No PI 5222), MeuCITx (UniProt Acc No P86401), GaTxl (UniProt Acc No P85066), Insectotoxin-I5 (UniProt Acc No P60270), Insectotoxin-Il (UniProt Acc No P15220); Bml2-b (UniProt Acc No Q9BJW4); BmK CT (UniProt Acc No Q9UAD0; particularly amino acids 25-59; resulting from the removal of signal peptide amino acid residues 1-24); AaCtx (UniProt Acc No P86436), MeuCITx-1 (UniProt Acc No P86402); Bsl4 (UniProt Acc No P59887); AmmP2 (UniProt Acc No P01498); BtlTx3 (UniProt Acc No P81761, particularly ammo acids 25-6; resulting from the removal of signal peptide amino acid residues 1-24) and amino acids 25-62; resulting from the removal of signal peptide amino acid residues 1-24 and pro-peptide amino acid residue 62.

The terms “antigen recognition domain,” “antigen recognition moiety,” “antigen binding domain,” “antigen binding moiety,” and the like, are used interchangeably herein. Similarly, the terms, “transmembrane domain,” “transmembrane moiety,” “transmembrane region,” and the like are used interchangeably; the terms “hinge domain,” “hinge moiety,” and “hinge region,” and the like are used interchangeably; the terms “intracellular signaling domain,” “signaling domain,” “signaling moiety,” “signaling region,” and the like are used interchangeably herein.

The phrase “therapeutically effective amount” or an “effective amount” in the context of the administration of an agent or composition to a subject, refers to an amount capable of having any detectable, positive effect on any symptom, aspect, or characteristic of a disease, disorder or condition, when administered to the subject; the agent or composition can be administered either alone or as part of a pharmaceutical composition and either in a single dose or as part of a series of doses. The therapeutically effective amount or effective amount can be ascertained by measuring relevant physiological effects, and it can be adjusted in connection with the dosing regimen and diagnostic analysis of the subject's condition, and the like. In reference to cancer or pathologies related to unregulated cell division, a therapeutically effective amount or an effective amount can refer to that amount which has the effect of (1) reducing the size of a tumor (i.e. tumor regression), (2) inhibiting (that is, slowing to some extent, preferably stopping) aberrant-cell division, for example cancer cell division, (3) preventing or reducing the metastasis of cancer cells, (4) relieving to some extent (or, preferably, eliminating) one or more symptoms associated with a pathology related to or caused in part by unregulated or aberrant-cellular division, including for example, cancer, (5) increasing the survival or life expectancy of the subject, and/or (6) decreasing the risk of relapse. An “effective amount” is also that amount that results in desirable PD and PK profiles and desirable immune cell profiling upon administration of the therapeutically active compositions of the invention. An “effective amount” is also an amount that achieves a recited effect or result; for example, an effective amount a chemotherapeutic agent that, alone or when in combination with another agent, can be an amount that reduces the size of a tumor and/or increases stress antigen expression on the tumor cells, and/or has a cytotoxic effect.

The terms “treating” or “treatment” of a disease (or a condition or a disorder) as used herein refer to inhibiting the disease (slowing or arresting its development), providing relief from the symptoms or side-effects of the disease (including palliative treatment), preventing or delaying recurrence, and causing regression of the disease. With regard to cancer, these terms also mean that the life expectancy of an individual affected with a cancer may be increased or that one or more of the symptoms of the disease will be reduced. With regard to cancer, “treating” also includes enhancing or prolonging an anti-tumor response in a subject.

As used herein any form of administration of a “combination”, “combined therapy” and/or “combined treatment regimen,” or “co-administration” or “co-administering,” or the like, refers to administration of at least two therapeutically active drugs or compositions (e.g., administration of the γδ T-cells and chemotherapeutic agent, or pharmaceutical compositions thereof), simultaneously or substantially simultaneously in either separate or combined formulations, or sequentially at different times separated by minutes, hours, days, weeks, or months, but in some way act together to provide the desired therapeutic response, for example, as part of the same treatment regimen.

The terms “enhance,” “enhancing” “enhanced”, or the like, as used herein, refers to an increase in the response or outcome referred to. For example, “enhancing cytotoxicity” refers to increasing cytotoxicity and “enhancing activation” refers to increasing activation. Similarly, enhanced persistence means increasing persistence. The term “enhancing” and the like can also encompass allowing a subject or tumor cell to improve its ability to respond to a treatment disclosed herein. An enhanced response can comprise an increase in responsiveness (cytotoxicity, activation and/or persistence) of at least about 5%, 10%, 15%, 20%, 25%, 30%, 35%, 40%, 45%, 50%, 55%, 60%, 65%, 70%, 75%, 80%, 85%, 90%, 95% or 98% or more. The enhanced responsiveness can encompass enhanced cytotoxicity to the cancer or tumor and/or enhanced T-cell activation and/or enhanced persistence.

Enhanced activation and/or enhanced cytotoxicity displayed or exhibited by the multivalent CLTX-CAR γδ T-cells (or composition thereof), for example, divalent CLTX-CAR γδ T-cells (or composition thereof), can encompass a greater than additive effect or a synergistic effect as compared to the additive effect that would be predicted based on the activation or cytotoxicity of a comparable sCLTX-CAR or a composition thereof. As defined herein, a “synergistic effect” is a greater than additive effect. An additive effect is considered the baseline for determining synergy and it is the effect that is theoretically expected or predicted based on the combination when synergy is not present (Roell et al. (2017), Front. Pharmacol. 8(158); https://doi.org/10.3389/fphar.2017.00158; the contents of which are expressly incorporated by reference herein). For example, when the multivalent CLTX-CAR γδ T-cells (or composition thereof) comprise only two CLTX peptides in the extracellular antigen binding domain, a synergistic or greater than additive effect on cytotoxicity or activation is greater than two-times the cytotoxicity or activation of a comparable sCLTX-CAR or a composition thereof. In certain embodiments, the greater than additive or synergistic effect of the dCLTX-CAR or a composition thereof is more than 2-fold. In certain specific embodiments, the greater than additive or synergistic effect of the dCLTX-CAR or a composition thereof is at least about 2.5-fold (or at least about 2.5 times), at least about 3-fold (or at least about 3 times), at least about 3.5-fold (or at least about 3.5 times), at least about 4-fold (or at least about 4 times), and at least about 4.5-fold (or at least about 4.5 times) greater than that of a comparable sCLTX-CAR or composition thereof.

The terms “subject” and “patient” as used herein include humans, mammals (e.g., cats, dogs, horses, etc.), living cells, and other living organisms. A living organism can be a mammal. Typical patients are mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Preferably, a system includes a sample and a subject. The term “living host” refers to host or organisms noted above that are alive and are not dead. The term “living host” refers to the entire host or organism and not just a part excised (e.g., a liver or other organ) from the living host. In preferred aspects, the subject or patient is a human subject or patient.

The term “γδ T-cells” or “gamma delta T-cells” as used herein refers to a subset of T-cells that express a distinct T-cell receptor (TCR) on their surface. The majority of T-cells have a TCR composed of two glycoprotein chains called α- and β-TCR chains. In contrast, in γδ T-cells, the TCR is made up of one 7-chain and one 6-chain. This group of T-cells is usually much less common than αβ T-cells. γδ T-cells are unique amongst T-cell types in that they do not require antigen processing and MHC presentation of peptide epitopes. Furthermore, γδ T-cells are believed to have a prominent role in recognition of lipid antigens, and to respond to stress-related antigens such as MIC-A and MIC-B and other ligands of the NKG2D receptor.

Human γδ T-cells can also exhibit an antigen-presenting capacity. Similar to dendritic cells (DCs), blood Vγ9Vδ2 T-cells are able to respond to signals from microbes and tumors and prime CD4⁺ and CD8⁺ T-cells. γδ T-APCs are believed to cross-present antigens directly to CD8⁺ T-cells. The intracellular protein degradation and endosomal acidification are significantly delayed in γδ T-cells in comparison to monocyte-derived DCs. The antigens are transported across IRAP (Insulin-Regulated Amino Peptidase)-positive early and late endosomes, and their processing consists of an export to the cytosol for degradation by the proteasome before being imported into an MHC-I-loading compartment. Activated γδ T-cells are able to phagocytose tumor antigens and apoptotic or live cancer cells possibly through the scavenger receptor CD36 in a C/EBPα (CCAAT/enhancer-binding protein α)-dependent mechanism and mount a tumor antigen-specific CD8⁺ T-cell response. γδ T-cells can also induce DC maturation through TNF-α production. Overall, γδ T-cells can process a wide range of antigens for presentation and stimulate other immune cells. Therefore, γδ T-cells implication in response to infections or cancer may be leveraged to design new strategies in order to improve clinical response of human γδ T-cell-based immunotherapy.

Increased tumor immunogenicity (e.g., increased upregulation of ligands for the NKG2D receptor), e.g., resulting from a chemotherapeutic agent or DDR inhibition is uniquely conducive to γδ T-cell-mediated tumor immunosurveillance, and ultimately tumor cell killing by γδ T-cells.

A cell composition or population of cells can be enriched for the γδ T-cells or the engineered γδ T-cells, for example. The term “enriched”, as used herein, refers to increasing the total percentage of one or more cytotoxic immune cell types present (e.g., γδ T-cells and/or NK cells) in a sample, relative to the total percentage of the same one or more cell types prior to enrichment, as disclosed herein. For example, a sample that is “enriched” for a for one or more types of cytotoxic immune cell may comprise between about 10% to 100% of the one or more cytotoxic immune cell types in the sample, whereas the total percentage of one or more of the cytotoxic immune cell types in a sample prior to enrichment was, for example, between 0% and 10%. Preferably, an enriched sample comprises at least 10%, 15%, 20%, 25%, 30%, 35%, 40%, 50%, 60%,70%, 80%, 90% or 100%, of one or more types of cytotoxic immune cell. Samples may be enriched for one or more cell types using standard techniques, for example, flow cytometry techniques. The term “highly enriched”, as used herein, refers to increasing the total percentage of one or more cytotoxic immune cell types in a sample such that the one or more cytotoxic immune cell types may comprise between at least about 70% to about 100% of the cytotoxic immune cell type in the sample, whereas the total percentage of that same type of cytotoxic immune cell prior to enrichment was, for example, between 0% and 10%. Preferably, a highly enriched sample comprises at least 60%, 65%, 70%, 75%, 80%, 85%, 90%, 95%, 99% or more of one or more types of cytotoxic immune cell. Samples may be highly enriched for one or more cell types using standard techniques, for example, flow cytometry techniques.

A cell composition or population can comprise an expanded population of γδ T-cells or the engineered γδ T-cells, or any subset thereof for example. The terms “expanded” and “expansion” as used herein with regard to expansion of one or more cytotoxic immune cells in a sample means to increase in the number of one or more cytotoxic immune cells in a sample by, for example about at least 2-fold, preferably by about 5-fold, preferably by at least 10-fold, preferably about at least 50-fold or more. Expansion of a cytotoxic immune cell population can be accomplished by any number of methods as are known in the art. For example, T-cells can be rapidly expanded using non-specific T-cells receptor stimulation in the presence of feeder lymphocytes and either interleukin-2 (TL-2) or interleukin-15 (IL-15), with TL-2 being preferred. The non-specific T-cells receptor stimulus can include around 30 ng/ml of OKT3, a mouse monoclonal anti-CD3 antibody (available from ORTHO-MCNEIL®, Raritan, N.J.). Alternatively T-cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, e.g., 0.3 μM MART-1:26-35 (27 L) or gp100:209-217 (210M), in the presence of a T-cell growth factor, such as 300 IU/ml IL-2 or IL-15, with IL-2 being preferred. Methods of expanding γδ T-cells have been described, for example, in WO2017035375 and WO2011053750; the contents of which are expressly incorporated by reference herein.

The γδ T-cells can also be derived from human induced pluripotent stem cells (hiPSCs). The pluripotent stem cells can, for example, be isolated from the patient having the cancer. In other aspects, the pluripotent stem cells may be isolated from a source other than the patient with cancer. The optionally enriched and/or optionally expanded compositions comprising γδ T-cells can also comprise natural killer (NK) cells and optionally further comprise other immunocompetent cells including but not limited to monocytes, macrophages and dendritic cells. Methods for generating γδ T-cells from induced pluripotent stem cells has been described, for example, in Watanabe et al. 2017, Stem Cells Transl Med 7(1): 34-44 and Zeng et al. (2019), PLoS One 14(5): e0216815; the contents of each of which are expressly incorporated by reference herein.

The terms “isolated’ and “isolated population” of cells as used herein refers to a cell or a plurality of cells removed from the tissue or state in which they are found in a subject. The terms may further include cells that have been separated according to such parameters as, but not limited to, cell surface markers, a reporter marker such as a dye or label.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein, a polypeptide, or a portion or fragment thereof.

The term “vector” as used herein refers to a polynucleotide comprised of single strand, double strand, circular, or supercoiled DNA or RNA. A typical vector may be comprised of the following elements operatively linked at appropriate distances for allowing functional gene expression; replication origin, promoter, enhancer, 5′ mRNA leader sequence, ribosomal binding site, nucleic acid cassette, termination and polyadenylation sites, and selectable marker sequences. One or more of these elements may be omitted in specific applications. The vector may also contain a nucleic acid cassette, which can include a restriction site for insertion of the nucleic acid sequence to be expressed. In a functional vector the nucleic acid cassette contains the nucleic acid sequence to be expressed including translation initiation and termination sites. A vector is constructed so that the particular coding sequence (for example, a coding sequence for a multivalent CLTX-CAR of the present disclosure) is located in the vector with the appropriate control sequences, the positioning and orientation of the coding sequence with respect to the control sequences being such that the coding sequence is operably linked and/or is transcribed “under the control” of the control sequences. Modification of the sequences encoding the particular protein of interest may be desirable to achieve this end. For example, in some cases it may be necessary to modify the sequence so that it may be operably linked to the control sequences with the appropriate orientation or to maintain the reading frame. The control sequences and/or other regulatory sequences may be ligated to the coding sequence prior to insertion into a vector. Alternatively, the coding sequence can be cloned directly into an expression vector that already contains the control sequences and an appropriate restriction site that is in reading frame with and under regulatory control of the control sequences.

The term “promoter” as used herein refers to the DNA sequence that determines the site of transcription initiation from an RNA polymerase. A “promoter-proximal element” may be a regulatory sequence within about 200 base pairs of the transcription start site.

The term “recombinant cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant-cell can be a single eukaryotic cell, or a single prokaryotic cell, or a mammalian cell. The recombinant-cell may harbor a vector that is extragenomic. An extragenomic nucleic acid vector does not insert into the cell's genome. A recombinant-cell may further harbor a vector or a portion thereof that is intragenomic. The term “intragenomic” defines a nucleic acid construct incorporated within the recombinant-cell's genome.

The terms “recombinant nucleic acid” and “recombinant DNA” as used herein refer to combinations of at least two nucleic acid sequences that are not naturally found in a eukaryotic or prokaryotic cell. The nucleic acid sequences include, but are not limited to, nucleic acid vectors, gene expression regulatory elements, origins of replication, suitable gene sequences that when expressed confer antibiotic resistance, protein-encoding sequences, and the like. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

The terms “operably” or “operatively linked” as used herein refer to the configuration of the coding and control sequences so as to perform the desired function. Thus, control sequences operably linked to a coding sequence are capable of effecting the expression of the coding sequence. A coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The terms “heterologous” and “exogenous” as they relate to nucleic acid sequences such as coding sequences and control sequences denote sequences that are not normally-associated with a region of a recombinant construct or with a particular chromosomal locus, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host-cell transformed with a construct, which is not normally present in the host-cell, would be considered heterologous for purposes of this invention.

Preferably, the promoter will be modified by the addition or deletion of sequences, or replaced with alternative sequences, including natural and synthetic sequences as well as sequences that may be a combination of synthetic and natural sequences. Many eukaryotic promoters contain two types of recognition sequences: the TATA box and the upstream promoter elements. The former, located upstream of the transcription initiation site, is involved in directing RNA polymerase to initiate transcription at the correct site, while the latter appears to determine the rate of transcription and is upstream of the TATA box. Enhancer elements can also stimulate transcription from, linked promoters, but many function exclusively in a particular cell type. Many enhancer/promoter elements derived from viruses, e.g., the SV40, the Rous sarcoma virus (RSV), and CMV promoters are active in a wide array of cell types, and are termed “‘constitutive” or ‘“ubiquitous.’” The nucleic acid sequence inserted in the cloning site may have any open reading frame encoding a polypeptide of interest, with the proviso that where the coding sequence encodes a polypeptide of interest, it should lack cryptic splice sites that can block production of appropriate mRNA molecules and/or produce aberrantly spliced or abnormal mRNA molecules.

The termination region that is employed primarily will be one of convenience, since termination regions appear to be relatively interchangeable. The termination region may be native to the intended nucleic acid sequence of interest, or may be derived from another source.

The term “targeted therapy”, as used herein, refers to any therapeutic molecule that targets any aspect of the immune system.

The terms “transformation”, “transduction” and “transduction” all denote the introduction of a polynucleotide into a recipient-cell or cells.

The invention provides an engineered γδ T-cell that expresses a multivalent CLTX chimeric antigen receptor (CLTX-CAR) and that express a survival factor, wherein the survival factor is a DNA, RNA, or polypeptide that confers resistance to a chemotherapeutic agent. The invention additionally provides an engineered γδ T-cell that expresses a multivalent CLTX chimeric antigen receptor (CLTX-CAR) and a polypeptide that confers resistance to a chemotherapeutic agent, wherein the γδ T-cell comprises a single vector that directs the expression of the CLTX-CAR and the polypeptide that confers resistance to a chemotherapeutic agent. As described above, the antigen binding domain of the multivalent CLTX-CAR comprises at least two CLTX peptides, wherein the at least two CLTX peptides are attached by a linker peptide; optionally, the linker peptide is 1-30 amino acids in length or the linker peptide is less than 15 amino acids in length. In certain aspects, the multivalent CLTX-CAR comprises two CLTX peptides (or in other words, “only two CLTX peptides”), three CLTX peptides (or in other words, “only three CLTX peptides”), or four CLTX peptides (or in other words, “only four CLTX peptides”). In this context, “only two CLTX peptides” and the like means that the antigen recognition domain does not comprise more than two CLTX peptides but may comprise additional amino acids, peptides, linker peptides, etc. so long as the number of CLTX peptides in the antigen recognition domain is two. Such CLTX-CAR(s) may further comprise additional moieties or domains in the extracellular domain. The multivalent CLTX-CARs described herein can further comprise a transmembrane domain, a hinge domain, and optionally at least one intracellular signaling domain, as well as a co-stimulatory domain. In certain aspects, the multivalent CLTX-CARs comprise a transmembrane domain, a hinge domain, at least one intracellular signaling domain, as well as at least one co-stimulatory domain. In further aspects, the multivalent or divalent CLTX-CARs comprise a transmembrane domain, a hinge domain, and does not include an intracellular signaling domain. Multivalent CLTX-CARs in accordance with the invention can have the following structure: i) an extracellular domain (also referred to herein as an “ectodomain”) comprising an antigen recognition domain/moiety comprising at least two CLTX peptide (or at least two of the following: a CLTX peptide, a functional variant of CLTX peptide, or a CLTX-like peptide), ii) a hinge domain; iii) a transmembrane domain and iii) an intracellular signaling domain (the intracellular signaling domain is part of the “endodomain” or the functional end of the receptor). Certain multivalent CLTX-CARs can comprise the following: i) an extracellular domain (also referred to herein as an “ectodomain”) comprising an antigen recognition domain/moiety comprising at least two CLTX peptide (or at least two of the following: a CLTX peptide, a functional variant of CLTX peptide, or a CLTX-like peptide), ii) a hinge domain; iii) a transmembrane domain and iv) an endodomain that does not include a signaling domain. In addition, certain divalent CLTX-CARs herein can comprise i) an extracellular domain (also referred to herein as an “ectodomain”) comprising an antigen recognition domain/moiety comprising only two CLTX peptides (or two of the following: a CLTX peptide, a functional variant of CLTX peptide, or a CLTX-like peptide), ii) a hinge domain; iii) a transmembrane domain and iv) an endodomain that does not include a signaling domain. In further aspects, certain divalent CLTX-CARs herein can comprise i) an extracellular domain (also referred to herein as an “ectodomain”) comprising an antigen recognition domain/moiety comprising only two CLTX peptides (or two of the following: a CLTX peptide, a functional variant of CLTX peptide, or a CLTX-like peptide), ii) a hinge domain; iii) a transmembrane domain and iv) an endodomain that includes a co-stimulatory domain but that does not include a signaling domain.

In certain embodiments, a peptide linker from 1 to 30 amino acids can be present in the CLTX-CAR to separate the various domains of the CAR. In yet other aspects, the peptide linker is less than 15 amino acids in length. For example, a peptide linker may be present between the antigen recognition domain/moiety and other domains which may be present in the extracellular domain, between the antigen recognition domain/extracellular domain and the hinge domain, between the hinge domain and the transmembrane domain, or between the transmembrane domain and the intracellular signaling domain. A peptide linker can be present between all domains or only between a portion of the domains/moieties. Furthermore, when the intracellular signaling domain comprises more than one element, a linker peptide may be present between some or all of the individual elements in the endodomain. Each linker peptide in the multivalent CLTX-CAR can be the same or can be different. An exemplary linker peptide that can link (or be positioned between) two CLTX peptides (or at least two of the following: a CLTX peptide, a functional variant of CLTX peptide, or a CLTX-like peptide) can be 30 amino acids in length or less, 20 amino acids in length or less, or 15 amino acids in length or less. Non-limiting examples of such linker peptides are FLAG, influenza virus haemagglutinin (HA), c-myc, polyHis; Strep tags, Strep II tags, FLAG tags, glutathione S-transferase (GST) tags, green fluorescent protein (GFP) tags, hemagglutinin A (HA) tags, histidine (His) tags, luciferase tags, maltose-binding protein (MBP) tags, c-Myc tags, protein A tags, protein G tags, a human serum albumin (HSA), or influenza virus haemagglutinin. In certain aspects, the peptide linker is c-myc (for example, having the amino acid sequence of EQKLISEEDL (SEQ ID NO: 2) or FLAG (for example, having the amino acid sequence of DYKDDDDK (SEQ ID NO: 3). Another example of a linker peptide is (GSSS)ₙ, wherein n is an integer from 1 to 10. In yet another embodiment, the linker peptide is HA, for example, having an amino sequence of GLFGAIAGFIENG (SEQ ID NO: 14) or EGMIDGWYG (SEQ ID NO: 15).

The intracellular signaling domain of the CLTX-CAR of the invention is responsible for activation of at least one of the normal effector functions of the host-cell in which the CLTX-CAR is placed. The term “effector function” refers to a specialized function of a differentiated cell. When the host-cell is an immune effector cell, the intracellular signaling domain of the CLTX-CAR of the invention is responsible for activation of at least one of a normal immune effector function. Immune effector function of a T-cell, for example, may be cytolytic activity or helper activity, including, but not limited to, the secretion of cytokines. Thus the term “‘intracellular signaling domain” refers to the portion of a CLTX-CAR that transduces the effector function signal and directs the ceil to perform a specialized function (for example, an effector function and/or an immune effector function). While usually the entire intracellular signaling domain will be employed, in many cases it will not be necessary to use the entire intracellular signaling domain. To the extent that a truncated portion of the intracellular signaling domain may find use, such truncated portion may be used in place of the intact signaling domain as long as such truncated portion still transduces the effector function/immune effector function signal. The term “intracellular signaling domain” is thus meant to include any truncated portion of the intracellular signaling domain sufficient to transduce the effector function/immune effector function signal. Examples of intracellular signaling domains include, but are not limited to, a signaling domain from the zeta chain of the T-cell receptor (CD3 zeta; CD247) or any of its homologs (e.g., eta, delta, gamma, or epsilon), MB1 chain, B29, FcRIII, FcRI, and combinations of signaling and/or costimulatory molecules, such as CD3 zeta chain and CD28, CD27, 4-IBB, DAP-10, OX40, and combinations thereof, as well as other similar molecules and fragments as well as mutations to the foregoing, such as modifying the immunoreceptor tyrosine-based activation motif(s) (ITAMs). In certain embodiments, the signaling domain comprises a CD3 zeta sequence, which may be represented by the sequence: RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQ ALPPR (SEQ ID NO: 4). Intracellular signaling portions of other members of the families of activating proteins can be used, such as FcγRIII and FcRI. One of skill in the art will be able to determine the corresponding signaling domains. Furthermore, any of the signaling domain sequences may contain from 1 to 5 amino acid modifications, which may be selected as discussed herein.

In certain aspects, the endodomain does not include an intracellular signaling domain.

In certain aspects, the intracellular signaling domain or endodomain of a CLTX-CAR comprises a sequence encoding a costimulatory signaling domain. For example, the intracellular signaling domain or endodomain can comprise a sequence encoding a primary signaling domain and a sequence encoding a costimulatory signaling domain. In certain embodiments, the costimulatory domain is a functional signaling domain from 41BB, OX40 and/or CD28. A costimulatory domain from OX40 can have the sequence:

ALYLLRRDQRLPPDAHKPPGGGSFRTPIQEEQADAHSTLAKI (SEQ ID NO: 5). A costimulatory domain from CD28 can have the sequence.
RSKRSRLLHSDYMNMTPRRPGPTRKHYQPYAPPRDFAAYRS (SEQ ID NO: 6). A costimulatory domain from 4IBB can have the sequence.
KRGRKKLLYIFKQPFMRPVQTTQEEDGCSCRFPEEEEGGCEL (SEQ ID NO: 7). Preferably, the encoded costimulatory signaling domain comprises a functional signaling domain of a protein chosen from one or more of CD27, CD28, 4-1BB (CD137), OX40, C.D30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8ct, CD8fi, IL2Rp, IL2Ry, IL7Ra, ITGA4, VLA1, CD49a, ITGA4, IA4, CD49D, ITGA6, VLA-6, CD49f, ITGAD, CDIId, ITGAE, CD103, ITGAL, CD18, LFA-1, ITGAM, CD1 lb, ITGAX, CD1 lc, 1TGB I, CD29, ITGB2, CD18, LFA-1, ITGB7, TNFR2, TRANCE/RA KL, DNAM1 (CD226), SLAMF4 (CD 244. 2B4), CD84, CD96 (Tactile), CEACAMI, CRTAM, Ly9 (CD229), CD 160 (BY55), PSGL1, CD 100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, NKp44, NKp30, NKp46, or NKG2D. One of skill in the art will be able to determine the corresponding transmembrane regions from these polypeptides. Furthermore, any of the costimulatory domain sequences may contain from 1 to 5 amino acid modifications, which may be selected as discussed herein. In certain embodiments, the signaling domain comprises CD3 zeta-CD28-OX40, CD3 zeta-4IBB, or CD28-41BB and CD3 zeta-CD28-41BB.

The extracellular domain comprising the antigen recognition domain can be linked to the intracellular signaling domain via an extracellular spacer (also referred to herein as an extracellular hinge domain) and/or a transmembrane domain. The extracellular antigen binding domain and the transmembrane domain can be linked by an extracellular hinge domain or an extracellular spacer sequence. Preferably, the extracellular spacer or extracellular hinge domain sequence comprises one or more of a hinge region and/or a portion of an immunoglobulin heavy chain constant region (which may comprise CH1, a linker region, CH2 and/or CH3 domains) or any combination thereof, of a human immunoglobulin, i.e. IgA, IgD, IgE, IgG, and IgM. In certain embodiments, extracellular spacer or hinge domain comprises all or a portion of the hinge region of human IgD. In certain embodiments, extracellular spacer or hinge comprises all or a portion of the hinge region of human IgG1. In certain embodiments, the extracellular spacer or hinge comprises all or a portion of the hinge region of human IgD and all or a portion of the hinge region of human IgGl. In certain embodiments, the extracellular spacer or hinge comprises all or a portion of the hinge region of human IgD and all or a portion of the CH2 and CH3 domains of the heavy-chain constant region of human IgG1. In certain embodiments, the extracellular spacer or hinge comprises all or a portion of the hinge region of human IgD, all or a portion of the hinge region of human IgG1 and all or a portion of the CH2 and CH3 domains of the heavy chain constant region of human IgG1. In certain embodiments, the extracellular spacer or hinge comprises all or a portion of the hinge region of human IgG1 and all or a portion of the CH2 and CH3 domains of the heavy chain constant region of human IgG1. In certain embodiments, extracellular spacer or hinge comprises all of the hinge region of human IgD, all or a portion of the hinge region of human IgG1 and the heavy chain constant region comprises all or a portion of the CH2 and CH3 domains of human IgG1. Preferably the hinge region amino acid sequence comprises the hinge region amino acid sequence from an immunoglobulin, such from IgD or IgG1, wherein the amino acid sequence comprises from 1 to 5 amino acid modifications, which may be selected as discussed herein. Preferably, the CH2 and CH3 domains of the heavy chain constant region comprises the CH2 and CH3 domain immunoglobulin heavy chain constant region amino add sequence from an immunoglobulin, such from IgG 1, wherein the amino acid sequence comprises from 1 to 5 amino acid modifications, which may be selected as discussed herein. In other aspects, the extracellular spacer or the extracellular hinge domain comprises the hinge region of a protein selected from the group consisting of CD8a, CD28, CD137, or a combination thereof. In certain aspects, the extracellular spacer or the extracellular hinge domain comprises the hinge region of CD8a. In any of the foregoing, the extracellular spacer may further comprise a linker, such as a linker having the sequence of Ser-Gly-Gly-Gly (SEQ ID NO: 8) or Ser-Gly-Gly-Gly-Gly (SEQ ID NO: 9), which may be present having from 1 to 10 copies, linking the extracellular spacer to the extracellular antigen binding domain.

In certain embodiments, the antigen recognition domain is linked to the transmembrane domain via a flexible linker. The flexible linker may be present in addition to the extracellular spacer or instead of the extracellular space described herein. In certain embodiments, the extracellular domain/antigen recognition domain is linked to the extracellular spacer via a flexible linker. Preferably, the flexible linker comprises, for example, glycine and serine. Preferably, the flexible linker is comprised of a polypeptide having the sequence of SEQ ID NO: 10 (Ser-Gly-Gly-Gly)ₙ or SEQ ID NO: 8 (Ser-Gly-Gly-Gly-Gly) wherein n is an integer from 1 to 10. Preferably, each flexible linker is a polypeptide comprising from about 1-25 amino acids, preferably about 1-15 amino acids, preferably about 1-10 amino acids, preferably about 4-24 amino acids, preferably about 5-20 amino acids, preferably about 5-15 amino acids and preferably about 5-12 amino acids. Preferably, the linker is (Ser-Gly-Gly-Gly)ₙ wherein n is 3.

The CLTX-CAR of the invention can comprise a transmembrane domain that corresponds to, or is derived or obtained from, the transmembrane domain of any molecule known in the art. For example, the transmembrane domain can correspond to that of a CD8 molecule or a CD28 molecule. CD8 is a transmembrane glycoprotein that serves as a co-receptor for the T-cell receptor (TCR) and is expressed primarily on the surface of cytotoxic T-cells. The most common form of CD8 exists as a dimer composed of a CD8 and CD80 chain. CD28 is expressed on T-cells and provides co-stimulatory signals required for T-cell activation. A transmembrane domain from a CD8 polypeptide may have the sequence IYIWAPLAGTCGVLLLSLVITLYC (SEQ ID NO: 11), particularly amino acids 1-21, 1-23 or 1-24 of SEQ ID NO: 13). CD28 is the receptor for CD80 (B7.1) and CD86 (B7.2). A transmembrane domain from a CD28 polypeptide may have the sequence FWVLVVVG GVLACYSLLVTVAFIIFWV (SEQ ID NO: 12). Preferably, the CD8 and CD28 are human. Preferred transmembrane domains of the CLTX-CARs of the invention include, but are not limited to, all or a portion of a transmembrane domain from a polypeptide selected from: an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDIIa, CD18), ICOS (CD278), 4-IBB (CD137), GITR, CD40, BAFFR, HVEM (LIGHTR), SLAMF7, NKp80 (KLRFI), CD160, CDI9, IL2Rβ, 1L2Rγ, IL7Rα, ITGA1, VLA1, CD49a, ITGA4, IA4, CD49D, ITGA6, VLA-6, CD49f, ITGAD, CDIId, ITGAE, CD 103, ITGAL, CD1 la, LFA-1, ITGAM, CDllb, ITGAX, CD1 lc, ITGBl, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD 160 (BY55), PSGLI, CD100 (SEMA4D), SLAMF6 (NTB-A, Lyl08), SLAM (SLAMFI, CD 150, IPO-3), BLAME (SLAMF8), SELPLG (CD 162), LTBR, PAG/Cbp, NKp44, NKp30, NKp46, NKG2D, and/or NKG2C. One of skill in the art will be able to determine the corresponding transmembrane regions from these polypeptides.

The multivalent CLTX-CAR can comprise any one of the aforementioned transmembrane domains and any one or more (e.g., 1, 2, 3, or 4) of the aforementioned intracellular T-cell signaling domains in any combination and any of the aforementioned hinge domain and any of the aforementioned co-stimulatory domains. For example, a CLTX-CAR can comprise a CD28 transmembrane domain and intracellular T-cell signaling domains of CD28 and CD3zeta. Furthermore, any of the transmembrane domain sequences may contain from 1 to 5 amino acid modifications, which may be selected as discussed herein.

An exemplary CLTX-CAR of the invention can comprise an antigen recognition domain comprising at least two CLTX peptides (or at least two of the following: a CLTX peptide, a functional variant of CLTX peptide, or a CLTX-like peptide). Another exemplary CLTX CAR comprises antigen recognition with comprises two CLTX peptides. The CLTX-CAR can further comprises one or more of the following: an optional linker linking the antigen recognition domain to the hinge domain; a hinge domain comprising all or a portion of a hinge region of CD8a, CD28, or CD137; preferably, the hinge region of CD8a; a transmembrane region from CD28; and/or an optional intracellular signaling region (or endodomain) comprising at least one signaling domain; preferably, CD3zeta, and an optional costimulatory signaling domain as described herein; preferably, the CD28 and/or the 4-1BB co-stimulatory domains. In yet further aspects, the multivalent CLTX-CAR can comprise an extracellular signal peptide. For example, the signal peptide can be the signal peptide of a protein selected from the group consisting of CD8a, CD28, GM-CSF, CD4, CD137, or a combination thereof. In certain aspects, the CLTX-CAR comprises only two CLTX peptides or only three CLTX peptides in the antigen binding domain.

In certain aspects, the multivalent CLTX-CAR (e.g., a divalent CLTX-CAR) does not comprise or include an intracellular signaling domain. Such a multivalent CLTX-CAR can comprise an extracellular domain (also referred to herein as an “ectodomain”) comprising an antigen recognition domain/moiety comprising at least two CLTX peptides (or at least two of the following: a CLTX peptide, a functional variant of CLTX peptide, or a CLTX-like peptide). In certain aspects, the extracellular domain comprises two CLTX peptides. In yet further non-limiting examples, the multivalent CLTX-CAR can further comprise a hinge domain and a transmembrane domain. In yet additional embodiments, the multivalent CLTX-CAR does not comprise or include a CD3 zeta (also referred to herein as CD3z or CD3ζ) signaling domain. Without wishing to bound by theory, the absence of a signaling domain in the CLTX-CAR γδ T cells may mitigate activation-induced cell death (AICD) which increases the persistence of the CLTX-CAR γδ T cells and thus prolong their effects. The multivalent CLTX-CAR that does not comprise or include an intracellular signaling domain may or may not include a co-stimulatory domain. Without wishing to be bound by theory, the co-stimulatory domain (e.g., the CD28 co-stimulatory domain) can act as an intracellular anchor, to stabilize the construct within the cellular membrane and/or to enhance or prolong cell surface expression.

The invention additionally includes a nucleic acid or a vector comprising the multivalent CLTX-CAR or a divalent CTLX-CAR as described herein. The nucleic acid or vector can comprise:
i. an extracellular antigen-binding domain comprising at least two CLTX peptides and wherein the at least two CLTX peptides are attached by a linker peptide; optionally, the linker peptide is 30 amino acids or less in length or wherein the linker peptide is less than 15 amino acids in length;
ii. a transmembrane domain;
iii. an extracellular hinge domain that attaches the transmembrane domain to the extracellular antigen-binding domain;
iv. optionally, an intracellular signaling domain; and v. optionally, a co-stimulatory domain.
   In certain aspects, the nucleic acid or vector further encodes a DNA, RNA or polypeptide that confers resistance to a chemotherapeutic agent as described herein. In yet additional aspects, nucleic acid or vector encodes a polypeptide that confers resistance to a chemotherapeutic agent.

In further aspects, the nucleic acid or vector encodes a self-cleaving peptide between the multivalent CLTX-CAR and the polypeptide. Example of self-cleaving peptides include, for example, porcine teschovirus-1 2A (P2A) sequence, thosea asigna virus 2A (T2A), equine rhinitis A virus 2A (E2A), cytoplasmic polyhedrosis virus (BmCPV 2A), and flacherie virus (BmIFV 2A) of B. mori. In certain aspect, the nucleic acid or vector encodes a P2A sequences between the multivalent CLTX-CAR and the survival polypeptide such as MGMT.

Specific multivalent CLTX-CARs include, for example, an antigen binding domain comprising two CLTX peptides separated by a linker peptide, a CD8a hinge domain, a CD28 co-stimulatory domain, a CD3ζ signaling domain and MGMT. In yet further embodiments, the multivalent CLTX-CARs include, for example, an antigen binding domain comprising two CLTX peptides separated by a linker peptide, a CD8a hinge domain, a CD28 co-stimulatory domain, no signaling domain and MGMT. Specific multivalent CLTX-CARs include, for example, an antigen binding domain comprising two CLTX peptides separated by a c-myc or Flag peptide, a CD8a hinge domain, a CD28 co-stimulatory domain, a CD3ζ signaling domain and MGMT. In yet further embodiments, the multivalent CLTX-CARs include, for example, an antigen binding domain comprising two CLTX peptides separated by a c-myc or Flag peptide, a CD8a hinge domain, a CD28 co-stimulatory domain, no signaling domain and MGMT. An extracellular c-myc or Flag peptide or other protein tag can be included for CAR-T detection and/or enrichment.

A multivalent CLTX-CAR according to the present invention can be produced by any means known in the art, though preferably it is produced using recombinant DNA techniques. A nucleic acid sequence encoding the several regions of the multivalent CLTX-CAR can prepared and assembled into a complete coding sequence by standard techniques of molecular cloning (genomic library screening, PCR, primer-assisted ligation, site-directed mutagenesis, etc.). The resulting coding region is preferably inserted into an expression vector and used to transform a suitable expression host-cell line, such as an immune effector cells, preferably a T lymphocyte cell line, and most preferably gamma delta T-cells (γδ-T cells) and stem cells that differentiate into these cells, can also be used. Preferably γδ-T cells are used as the host-cell line. As used herein, a “nucleic acid construct” or “nucleic acid sequence” is intended to mean a nucleic acid molecule, such as a DNA molecule, that can be transformed or introduced into an expression host-cell line, such as, but not limited to, a T-cell, and be expressed to produce a product (e.g., a chimeric receptor). Therefore, the invention further provides an isolated or purified nucleic acid sequence encoding the multivalent CLTX-CARs of the invention. “Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides. In the nucleic acid construct employed in the present invention, the promoter is operably linked to the nucleic acid sequence encoding a CLTX-CAR of the present invention, i.e., they are positioned so as to promote transcription of the messenger RNA from the DNA encoding the chimeric receptor. The promoter can be of genomic origin or synthetically generated. A variety of promoters for use in T-cells are well-known in the art. The promoter can be constitutive or inducible, where induction is associated with the specific cell type or a specific level of maturation, for example. Alternatively, a number of well-known viral promoters are also suitable. Promoters of interest include the β-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter associated with a different promote.

The invention is also directed to an engineered γδ T-cell that expresses a single CLTX chimeric antigen receptor (or a sCLTX CAR or 1×CLTX CAR) and a survival factor, wherein the survival factor is a polypeptide that confers resistance to a chemotherapeutic agent, wherein the γδ T-cell comprises a single vector that directs the expression of the single CLTX-CAR and the survival factor, and further wherein:
a. the CLTX-CAR comprises:
   i. an extracellular antigen-binding domain comprising one CLTX peptide,
   ii. an extracellular linker peptide, wherein the linker peptide is less than 30 amino acids in length, or 15 amino acids in length, wherein the linker peptide is located between the CTLX peptide and the transmembrane domain or between the CLTX peptide and the extracellular hinge domain;
   iii. a transmembrane domain;
   iv. an optional extracellular hinge domain that attaches the transmembrane domain to the extracellular antigen-binding domain;
   v. optionally, an intracellular signaling domain; and
   vi. optionally, a co-stimulatory domain.
      The invention also encompasses a population of the engineered γδ T-cells described herein. In certain aspects, the intracellular signaling domain is present. In additional aspects, the linker peptide is located directly or indirectly links the CTLX peptide to the transmembrane domain. In further aspects, the linker peptide directly or indirectly links the CLTX peptide and the extracellular hinge domain. In yet further aspects, the linker peptide is 15 amino acids in length. In certain aspects, the linker peptide is a Flag peptide, a myc peptide or an HA peptide. In certain embodiments, the single CLTX-CAR comprises a signaling domain and the linker peptide has enhanced activation as compared to an otherwise identical single CLTX-CAR without the linker peptide. The invention additionally encompasses pharmaceutical compositions comprising the γδ T-cell that expresses the single CLTX-CAR as well as methods of treating cancer or tumor as described herein.

The invention additionally includes an engineered γδ T-cell that expresses a single CLTX chimeric antigen receptor (or a sCLTX CAR or 1×CLTX CAR) and a survival factor, wherein the survival factor is a polypeptide that confers resistance to a chemotherapeutic agent, wherein the γδ T-cell comprises a single vector that directs the expression of the single CLTX-CAR and the survival factor, and further wherein:
a. the CLTX-CAR comprises:
   i. an extracellular antigen-binding domain comprising one CLTX peptide,
   ii. a transmembrane domain;
   iii. an optional extracellular hinge domain that attaches the transmembrane domain to the extracellular antigen-binding domain;
   iv. optionally, a co-stimulatory domain;

wherein the single CLTX-CAR does not include an intracellular signaling domain. The invention also encompasses a population of the engineered γδ T-cells described herein The invention also includes the single CTLX-CAR that comprises a co-stimulatory domain (e.g., a CD28 co-stimulatory domain) and does not comprise the intracellular signaling domain. The invention additionally encompasses pharmaceutical compositions comprising the γδ T-cell that expresses the single CLTX-CAR as well as methods of treating cancer or tumor as described herein.

The various manipulations for preparing the CLTX-CARs (e.g., the multivalent CTLX-CARs, the divalent CLTX-CARs, or the sCLTX-CARs) of the invention can be carried out in vitro and the CLTX-CAR chimeric construct can be introduced into vectors for cloning and expression in an appropriate host-cell using standard transformation or transfection methods.

Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like. Therefore, the invention comprises vectors encoding multivalent CLTX-CARs described herein or functional equivalents thereof.

As is well-known to one of skill in the art, various methods are readily available for isolating and expanding these cells from a subject. For example, using cell surface marker expression or using commercially available kits. It is contemplated that the chimeric construct can be introduced into the subject's own T-cells as naked DNA or in a suitable vector. Methods of stably transfecting T-cells by electroporation using naked DNA are known in the art. Naked DNA generally refers to the DNA encoding a chimeric receptor of the present invention contained in a plasmid expression vector in proper orientation for expression. Advantageously, the use of naked DNA reduces the time required to produce T-cells expressing the chimeric receptor of the present invention.

Therefore, the invention comprises host-cells containing (i.e., transformed or transduced with) vectors encoding multivalent CTX-CAR(s), divalent CLTX-CARs and sCLTX-CARs of the invention, as well as functional variants thereof. Preferably the host-cells are immune effector cells, preferably T-cells, a T lymphocyte cell line, and most preferably an autologous T lymphocyte cell line, a third party-derived T-cell line/clone, a transformed humoral or xenogenic immunologic effector cell line, for expression of the CLTX-CAR. Natural killer (NK) cells, macrophages, neutrophils, tumor-infiltrating-lymphocytes (TILs), lymphokine-activated killer (LAK) cells, memory T-cells, regulator}-T-cells, cytotoxic T lymphocytes (CTLs), gamma delta T-cells (γδ-T cells) and stem cells that differentiate into these cells, can also be used. Preferably γδ-T cells are used as the host-cell line. Once it is established that the transfected or transduced T-cell is capable of expressing the chimeric receptor as a surface membrane protein with the desired regulation and at a desired level, it can be determined whether the chimeric receptor is functional in the host-cell to provide for the desired signal induction. Subsequently, the transduced T-cells are reintroduced or administered to the subject to activate anti-tumor responses in the subject.

In one embodiment, the invention comprises host-cells, for example, γδ-T-cells, comprising (i.e., transformed or transduced with) one vector that encodes (i.e., directing the expression of) a multivalent CLTX-CAR(s), a divalent CLTX-CAR, or sCLTX-CAR of the present disclosure and a survival factor, such as a polypeptide that confers resistance to a chemotherapeutic agent as disclosed herein. Any CLTX-CAR of the present disclosure may be used. The γδ T-cells can naturally express a receptor for a stress-induced antigen (such as but not limited to, NKG2D); preferably, expression of the stress-induced antigen (to which the stress-induced antigen receptor) binds is increased by administration of the chemotherapeutic agent. For example, the γδ T-cells can naturally express NKG2D and as such can be utilized in a method of treatment comprising administration of a chemotherapeutic agent, wherein the administration of the chemotherapeutic agent increases the expression NKG2DL on tumor or cancer cells. In yet other aspects, the γδ-T-cells can comprise a vector (the same vector that encodes the CLTX-CAR or a different vector) that encodes (i.e., directing the expression of) a stress-induced antigen receptor (such as but not limited to, NKG2D).

As described above, the γδ T cells express a CLTX CAR, e.g., a multivalent CLTX-CAR, and further express a survival factor and/or have been treated with a survival factor, wherein the survival factor is a DNA, RNA or polypeptide that confers resistance to a chemotherapeutic agent. In certain aspects, the cell express the survival factor and the survival factor is a polypeptide that confers resistance (to the γδ-T-cell) to the chemotherapeutic agent allows the γδ-T-cells to survive in a treatment environment created by the chemotherapeutic agent and/or allows the γδ-T-cells to survive in the tumor environment comprising the chemotherapeutic agent. In preferred aspects, a single vector encodes the multivalent CLTX-CAR and the polypeptide that confers resistance to a chemotherapeutic agent. In certain specific embodiments, a single vector encodes the multivalent CLTX-CAR and a survival polypeptide selected from the group consisting of alkyl guanine transferase (AGT), P140K MGMT, O⁶ methylguanine DNA methyltransferase (MGMT), L22Y-DHFR, thymidylate synthase, dihydrofolate reductase, multiple drug resistance-1 protein (MDR1), 5′ nucleotidase II, dihydrofolate reductase, and thymidylate synthase. In yet additional aspects, the single vector encodes the multivalent CLTX-CAR and MGMT. In yet further aspects, the single vector encodes the multivalent CLTX-CAR and MGMT, wherein the multivalent CLTX-CAR comprises two CLTX peptides in the antigen recognition domain. In certain embodiments, the host-cell comprising the vector that directs the expression of the multivalent CLTX-CAR and the survival factor (and optionally a stress-induced antigen receptor) is an isolated or purified γδ T-cell. As discussed herein, the host-cells can be engineered to express a survival polypeptide that allows the host-cell, for example, the γδ T-cell to survive in a treatment environment created a chemotherapeutic agent). Such cells which express a survival polypeptide are referred to herein as drug resistant (DR) cells and their use in therapy is referred to herein as “drug resistant immunotherapy” (DRI). DR cells and DRI is described in WO 2011/053750, the teachings of which are hereby incorporated by reference into the present application. The survival polypeptide can be any polypeptide known in the art that provides resistance to a treatment regimen comprising a chemotherapeutic agent, and/or allows the cells comprising the survival polypeptide and the multivalent CLTX-CAR described herein to survive in a treatment environment created by the chemotherapeutic agent.

Exemplary chemotherapeutic agents are nucleoside-analog chemotherapy drug, alkylating agent, antimetabolite, antibiotic, topoisomerase inhibitor, mitotic inhibitor, differentiating agent, or hormone therapy agent and the survival factor provides resistance to the chemotherapeutic agent. In additional aspects, the chemotherapeutic agent is an alkylating agent. In certain embodiments, the survival polypeptide is MGMT, multidrug resistance protein 1 (MDRI), or 5′ nucleotidase II (NT5C2). In yet further aspects, the survival polypeptide is MGMT and the chemotherapeutic agent is an alkylating agent such as carmustine (BCNU), lomustine (CCNU), and temozolomide. In certain aspects, the chemotherapeutic agent is temozolomide (TMZ). In additional aspects, the survival polypeptide is MDR1 and the chemotherapeutic agent is an anthracycline, vinca alkaloids, epipodophyllotoxins, camptothecin, methotrexate (MTX), saquinavir, and mitoxantrone (MX) (Sodani et all. (2011). Multidrug resistance associated proteins in multidrug resistance. Chin J Cancer 31(2): 58-72). NT5C2 is a polypeptide known in the art to provide resistance to thiopurine chemotherapy (Tzoneva et al. (2013), Activating mutations in the NT5C2 nucleotidase gene drive chemotherapy resistance in relapsed ALL, Nat Med. 19(3): 368-371). Other survival polypeptide include, for example, a drug resistant variant of dihydrofolate reductase (L22Y-DHFR) and thymidylate synthase. In certain aspects, the survival polypeptide is MGMT. However, other survival factors may be used depending on the chemotherapeutic agent being co-administered, the nature of the treatment environment (i.e., what other treatment regimens are being given to the patient in combination with the cells compositions of the present disclosure).

In additional aspects, the chemotherapeutic agent is an alkylating agent; a metabolic antagonist; a DNA demethylating agent; a substituted nucleotide; a substituted nucleoside; an antitumor antibiotic; a plant-derived antitumor agent or a nitrosourea. Preferably the chemotherapeutic agent is selected from cisplatin; carboplatin; etoposide; methotrexate (MTX); trimethotrexate (TMTX); temozolomide; dacarbazine (DTIC), raltitrexed; S-(4-Nitrobenzyl)-6-thioinosine (NBMPR); 6-benzyguanidine (6-BG); a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; camptothecin; and a therapeutic derivative of any thereof. Preferably, the γδ T-cells have been genetically modified to encode alkyl guanine transferase (AGT), P140KMGMT, O⁶ methylguanine DNA methyltransferase (MGMT), L22Y-DHFR, thymidylate synthase, dihydrofolate reductase, or multiple drug resistance-1 protein (MDR1). Preferably, the γδ T-cells have been genetically modified to be resistant to at least two chemotherapeutic agents selected from: is an alkylating agent; a metabolic antagonist; a DNA demethylating agent; a substituted nucleotide; a substituted nucleoside; an antitumor antibiotic; a plant-derived antitumor agent and a nitrosurea. Preferably, the γδ T-cells have been genetically modified to be resistant to at least two chemotherapeutic agents selected from cisplatin; carboplatin; etoposide; methotrexate (MTX); trimethotrexate (TMTX); temozolomide; dacarbazine (DTIC), raltitrexed; S-(4-Nitrobenzyl)-6-thioinosine (NBMPR); 6-benzyguanidine (6-BG); a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; camptothecin; and a therapeutic derivative of any thereof. Preferably, the chemotherapeutic agent is TMZ, methotrexate, DTIC, BCNU, CCNU, MCNU, NMU or ENU.

A survival factor, including for example, the polypeptide that confers resistance to a chemotherapeutic agent (e.g., the chemotherapeutic agent being administered to the subject), can promote survival of the host cell expressing it in a treatment environment created by a chemotherapeutic agent, or survival in the presence of the chemotherapeutic agent when the host-cell survives in the presence of toxicity in the environment or the tumor microenvironment resulting from administration of the chemotherapeutic agent as part of the treatment.

Chemotherapeutic agents for use with DRI (and γδ T-cells expressing the polypeptide that confers resistance to the chemotherapeutic agent) include, but are not limited to: alkylating agents (e.g., cyclophosphamide, ifosfamide, melphalan); metabolic antagonists (e.g., methotrexate (MTX), 5-fluorouracil or derivatives thereof); DNA demethylating agents (also known as antimetabolites; e.g., azacitidine): a substituted nucleotide; a substituted nucleoside; antitumor antibiotics (e.g., mitomycin, adriamycin); plant-derived antitumor agents (e.g., vincristine, vindesine, TAXOL®, paclitaxel, abraxane); cisplatin; carboplatin; etoposide; and the like. Such agents may further include, but are not limited to, the anti-cancer agents trimethotrexate (TMTX); temozolomide (TMZ); raltitrexed; S-(4-Nitrobenzyl)-6-thioinosine (NBMPR); 6-benzyguanidine (6-BG); nitrosoureas (for example, bis-chloroethylnitrosourea, also known as BCNU and carmustine, lomustine, also known as CCNU, +/−procarbazine and vincristine (PCV regimen) and fotemustine); doxorubicin; cytarabine; camptothecin; and a therapeutic derivative of any thereof.

The engineered γδ-T cell as described herein can further express a suicide gene. A “suicide gene” as used herein refers to a mechanism by which the CLTX-CAR-expressing cells described herein may be eradicated from a subject administered with the cells or a composition thereof, for example, in order to protect against a cascading inflammatory response or off-target cytotoxicity. The suicide gene system can, for example, be a Herpes Simplex Virus Thymidine Kinase (HSVTK)/Ganciclovir (GCV) suicide gene system, an inducible Caspase suicide gene system (Budde et al., PLoS One 2013 8(12):82742), codon-optimized CD20 (Marin et al., Hum. Gene Ther. Meth. 2012 23(6)376-86), CD34, a truncated EGFR (Wang X, Chang W-C, Wong C W, et al. A transgene-encoded cell surface polypeptide for selection, in vivo tracking, and ablation of engineered cells. Blood. 2011; 118(5):1255-1263. doi:10.1182/blood-2011-02-337360), a truncated CD19, or polypeptide RQR8 (Philip et al, and WO2013153391A, which is hereby incorporated herein by reference). An additional example of a suicide gene is the τ-retrovirus SFG.iCaspase9.2A.DeltaCD19 which consists of iC9 linked, via a 2A-like sequence, to truncated human CD 19 that serves as selectable marker. AP1903-inducible activation of the Caspase 9 suicide gene is achieved by expressing a chimeric protein (iC9), fused to a drug-binding domain derived from human FK506-binding protein (FKBP). The iC9 is quiescent inside cells until exposure to API 903, which cross-links the FKBP domains, initiates iCasp9 signaling, and induces apoptosis of the gene-modified cells. The gene and API 903 is available from Bellicum Pharmaceuticals (Houston, Tex.).

DR γδ-T cells that express the CLTX-CAR or the multivalent CLTX-CAR of the invention, can be produced by incorporating a nucleic acid construct coding for and capable of expressing a CLTX-CAR described herein and optionally, can further express a DNA, RNA or polypeptide that confers resistance to a polypeptide, and optionally other elements (for example, a suicide gene and/or a receptor for a stress-induced antigen). In certain embodiments, a single nucleic acid construct codes for the multivalent CLTX-CAR and the polypeptide that confers resistance to the chemotherapeutic agent, as well as the additional optional elements (for example, a suicide gene and/or a receptor for a stress-induced antigen). In certain embodiments, separate nucleic acid constructs code for each the multivalent CLTX-CAR and the polypeptide that confers resistance to a chemotherapeutic agent, and the optional other elements (for example, a suicide gene and/or a receptor for a stress-induced antigen). In certain embodiments, a single nucleic acid construct codes for the multivalent CLTX-CAR and the polypeptide that confers resistance to a chemotherapeutic agent and one or more nucleic acid constructs codes for the additional optional elements (for example, a suicide gene and/or a receptor for a stress-induced antigen).

The γδ T-cell expressing the CLTX-CAR, e.g., the multivalent CLTX-CAR, can further expresses a receptor for a stress-induced antigen. In certain aspects, the γδ T-cell naturally expresses the stress-induced antigen, for example, NKGD2. In other aspects, the γδ T-cell is engineered to express the stress-induced antigen. In certain embodiments, the host-cell expressing a CLTX-CAR or a multivalent CTX-CAR of the present disclosure further comprises a gene encoding for the stress-induced antigen receptor, such as NKGD2. In certain embodiments, the stress-induced antigen receptor, including, but not limited to, the NKGD2 receptor is induced to an increased level on the γδ T-cell.

The host-cell, such as the γδ-T cells expressing the CTLX-CAR or the multivalent CLTX-CAR, for example, the γδ-T cells expressing the multivalent CLTX-CAR and the DNA, RNA or polypeptide that confers resistance to the chemotherapeutic agent, is administered as part of a composition. The composition can comprise the engineered 76-T cells and additional immune system cells. For example, the composition may comprise γδ T-cells expressing the multivalent CLTX-CAR as described herein, and can further comprise NK cells and/or ap T-cells. In certain aspects, the composition comprises the engineered γδ T-cells expressing a multivalent CLTX-CAR described herein and an additional immune system cell, wherein the 76 T-cells are present at greater than or equal to 50%, 60%, or 70% of the total cell population, for example, as determined by flow cytometry. In yet further aspects, the γδ T-cells are present at greater than or equal to 50%, 60%, or 70% of the total viable cell population, for example, as determined by flow cytometry. In certain embodiments, the composition comprises the engineered γδ T-cells and NK cells, wherein the γδ T-cells are present at greater than or equal to 50%, 60%, or 70% of the total cell population or the total viable cell population and the NK cells are present at less than or equal to 25% (for example, as determined by flow cytometry). In certain embodiments, the composition comprises the engineered γδ T-cells and ap T-cells, wherein the γδ T-cells are present at greater than or equal to 50%, 60%, or 70% of the total cell population or the total viable cell population, for example, as determined by flow cytometry. In additional aspects, the composition comprises the ap T-cells at less than or equal to 5% of the total cell population or the total viable cell population, for example, as determined by flow cytometry. In certain embodiments, the composition comprises the engineered γδ T-cells, ap T-cells and NK cells, wherein the γδ T-cells are present at greater than or equal to 50%, 60%, or 70% of the total cell population or the total viable cell population, for example as determined by flow cytometry. In certain embodiments, the ap T-cells are present at less than or equal to 5% of the total cell population or the total viable cell population, and the NK cells are present at less than or equal to 25% of the total cell population or the total viable cell population, as determined by flow cytometry.

Preferably, therapeutic compositions for administration to a patient comprising optionally enriched and/or optionally expanded population of γδ T-cells comprise about 5×10⁸ γδ T-cells/kg or less of a patient's weight. Preferably, therapeutic compositions for administration to a patient comprising optionally enriched and/or optionally expanded population of γδ T-cells comprise about 5×10⁷ γδ T-cells/kg or less of a patient's weight. Preferably, therapeutic compositions for administration to a patient comprising optionally enriched and/or optionally expanded population of γδ T-cells comprise about 5×10⁶ γδ T-cells/kg or less of a patient's weight.

Methods for isolating γδ T-cells either from a patient to be treated or from another source, as described, for example, by Lamb L. S. in U.S. Pat. No. 7,078,034, incorporated herein by reference in its entirety.

As described above, the use of the survival factor, including, for example, the polypeptide that confers resistance to a chemotherapeutic agent (such as the MGMT polypeptide), enables the compositions comprising the engineered γδ T-cells of the present disclosure (including a DR γδ T-cells) to survive in a treatment environment created by the chemotherapeutic agent at a time when the tumor is stressed. The stress effect on the tumor (e.g., by the chemotherapeutic agent) in certain embodiments increases the expression of stress antigens, which are recognized by receptors, such as the NKG2D receptor, on the γδ T-cells. The dual effect of inducing stress antigens and decreasing regulatory T-cells with chemotherapy significantly improve tumor reduction over either individual regimen. Gene modification (expressing the survival polypeptide) and/or treatment with a survival factor as described herein protects the compositions of the present disclosure from the lymphodepleting effects of a chemotherapy regimen, for example TMZ, and allows the cell compositions of the present disclosure specific access to the tumor via TAA combined with unimpaired T-cell cytotoxic function at the time that malignant-cells are maximally stressed by chemotherapy. The use of DRI in combination with a CLTX-CAR or a multivalent CLTX-CAR in accordance with the invention is referred to herein as “DRI CLTX-CAR” therapy, is believed to significantly prolong survival and reduce tumor burden and time to recurrence when compared with either chemotherapy (for example, TMZ) treatment alone or γδ T-cell infusion, for example, alone and do so without significant adverse systemic or neurologic consequences.

The compositions described herein can be delivered as a pharmaceutical composition, or made into an implant appropriate for administration in vivo, with appropriate carriers or diluents, which further can be pharmaceutically acceptable. The means of making such a composition or an implant have been described in the art. Where appropriate, the engineered γδ T-cells described herein can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed which does not ineffectuate the cells expressing the CLTX-CAR. Thus, desirably the cells expressing the multivalent CLTX-CAR as described herein can be made into a pharmaceutical composition containing a balanced salt solution, for example, Hanks' balanced salt solution, or normal saline. Therefore, the invention includes pharmaceutical compositions comprising γδ T-cells expressing a multivalent CLTX-CAR of the present disclosure, and specifically includes γδ T-cells expressing a multivalent CLTX-CAR and expressing the polypeptide that confers resistance to a polypeptide.

The pharmaceutical composition can be used alone or in combination with other well-established agents useful for treating cancer, for example, a chemotherapeutic agent as described herein. Whether delivered alone or in combination with other agents, the pharmaceutical composition of the present invention can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

The composition can be provided in unit dosage form wherein each dosage unit, e.g., an injection, contains a predetermined amount of the composition, alone or in appropriate combination with oilier active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject. Preferably, a therapeutically effective amount or sufficient number of the engineered γδ T-cells, administered alone or in combination with a therapeutic agent, is introduced into the subject such that a long-term, specific, response is established. In one embodiment, the response includes inhibition of cancer. In one embodiment, the response is the reduction in size of a tumor or elimination of tumor growth or regrowth or a reduction in metastasis to a greater degree than would otherwise result in the absence of the treatment with the engineered γδ T-cells or composition thereof. In certain aspects, the therapeutically effective amount results in at least about 5%, 10%, 20%, 30%, 40%, 50%, 60%, 70%, 80%, 90%, 95%, 98%, or 100% decrease in tumor size when compared that in the absence of the engineered CLTX-CAR. Accordingly, the therapeutically effective amount takes into account the route of administration and the number of engineered cells should be such that a sufficient number of so as to achieve the desired therapeutic response. Furthermore, the amounts of each γδ T-cells expressing a CLTX-CAR of the present disclosure or other cell included in the compositions described herein (e.g., the amount per each cell to be contacted or the amount per certain body weight) can vary in different applications. In certain non-limiting examples, the concentration of the cells can be sufficient to provide in the subject being treated at least from about 1×10⁵ to about 1×10¹⁰ host-cells, even more desirably, from about 1×10⁷ to about 5×10⁸ host-cells, although any suitable amount can be utilized either above, e.g., greater than 5×10^{s} cells, or below, e.g., less than 1×10⁷ cells. The dosing schedule can be based on well-established cell-based therapies or an alternate continuous infusion strategy can be employed.

These amounts provide general guidance to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on inter-individual differences in pharmacokinetics, drug disposition, and metabolism. One skilled in the art readily can make any necessary adjustments in accordance with the exigencies of the particular situation. Suitable doses for a therapeutic effect would be between about 10⁵ and about 10¹⁰ host-cells per dose, preferably in a series of dosing cycles. A preferred dosing regimen consists of four one-week dosing cycles of escalating doses, starting at about 1 (P ceils on Day 0, increasing incrementally up to a target dose of about 10¹⁰ cells by Day 5. Suitable modes of administration include intravenous, subcutaneous, intracavitary (for example by reservoir-access device), intraperitoneal, and direct injection into a tumor mass.

The cancer to be treated can be of neuroectodermal origin. In certain aspects, the cancer is a malignant glioma, melanoma, neuroblastoma, medulloblastoma or small cell lung carcinoma. The infused cells are able to kill tumor cells in the recipient. Unlike antibody therapies, host-cells expressing a CLTX-CAR are able to replicate in vivo resulting in long-term persistence that can lead to sustained tumor control. The invention also includes a cellular therapy where γδ-T cells are modified to transiently express a CLTX-CAR of the invention and the survival polypeptide, wherein the cells are infused to a recipient in need thereof. The infused cells are able to kill tumor cells in the recipient. Thus, in various aspects, the γδ-T cells administered to the patient, is present for less than one month, e.g., three weeks, two weeks, one week, after administration to the patient. In certain aspects, the cells administered to the patient, or their progeny, persist in the patient for at least four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, two years, three years, four years, or five years after administration of the host-cells to the patient.

In further aspects, γδ-T-cells can be a type of vaccine for ex vivo immunization and/or in vivo therapy in a mammal. In one aspect, the mammal is a human. With respect to ex vivo immunization, at least one of the following occurs in vitro prior to administering the host-cell or composition, including a pharmaceutical composition, comprising the host-cell into a mammal: i) expansion of the host-cells, ii) introducing a nucleic acid encoding the multivalent CLTX-CAR and the survival polypeptide to the host-cells and/or iii) cryopreservation of the cells expressing or capable of expressing the CLTX-CAR. Ex vivo procedures are well known in the art. Briefly, cells are isolated from a patient (e.g., a human) and genetically modified so as to express a CLTX-CAR of the present disclosure (i.e., transduced or transfected in vitro with a vector expressing a CLTX-CAR disclosed herein). The CLTX-CAR-modified host-cell can be administered to a patient to provide a therapeutic benefit. The patient is preferably a human and the CLTX-CAR-modified host-cell can be autologous with respect to the patient. Alternatively, the host-cells can be allogeneic, syngeneic or xenogeneic with respect to the patient.

The engineered γδ T-cells and the chemotherapeutic agent (e.g., the chemotherapeutic agent to which the survival factor confers resistance) can be co-administered. Such co-administration can encompass “simultaneous” or “concurrent delivery,” e.g., in the same or in separate compositions. In other aspects, co-administration encompasses separate administration but as part of the same treatment regimen. In certain aspect, the chemotherapeutic agent is administered before or concurrently with the engineered γδ T-cells. In additional aspects, the engineered γδ T-cells are co-administered with the chemotherapeutic agent, wherein the chemotherapeutic agent causes increased expression of a stress ligand (e.g, NKG2DL) on the tumor or cancer cells; for example, the chemotherapeutic agent is administered in an amount and in a manner/regiment resulting in increased express of the stress ligands. In certain aspects, the co-administration can be more effective than that of either treatment alone. The effect of the two treatments can be partially additive, wholly additive, or greater than additive. For example, co-administration can encompass administration of the γδ T-cells about 8 hours to about 72 hours after administration of the chemotherapeutic agent. In certain aspects, the engineered γδ T-cells are administered about 12 hours to about 36 hours after administration of the chemotherapeutic agent; for example, the engineered γδ T-cells are administered about 24 hours after administration of the chemotherapeutic agent. In yet further aspects, co-administration encompasses administering the engineered γδ T-cells and the same time or at substantially the same time as the chemotherapeutic agent. As used herein “substantially the same time” can encompass administration within the same treatment session.

The engineered γδ T-cells and the chemotherapeutic agent can be administered during periods of active disorder, or during a period of remission or less active disease.

In further aspects, an additional therapeutic agent is administered in addition to the γδ T-cells and the chemotherapeutic agent. When administered in combination, the γδ T-cells and the chemotherapeutic agent and optionally, the additional therapeutic agent, the amount or dosage of one or all of the foregoing, can be administered in an amount or dose that is higher, lower or the same than the amount or dosage of each agent used individually, e.g., as a monotherapy. In certain embodiments, the amount or dosage of one or all of the foregoing, is lower (e.g., at least 20%, at least 30%, at least 40%, or at least 50%) than the amount or dosage of each agent used individually, e.g., as a monotherapy. In other embodiments, the amount or dosage of one or all of the foregoing, that results in a desired effect (e.g., inhibition of cancer) is lower (e.g., at least 20%, at least 30%, at least 40%, or at least 50% lower) than the amount or dosage of each agent used individually, e.g., as a monotherapy, required to achieve the same therapeutic effect.

The engineered γδ T-cells and the chemotherapeutic agent can be administered in combination with an additional therapeutic treatment, such as, but not limited to, surgery, chemotherapy (e.g., an additional chemotherapeutic agent different from the chemotherapeutic agent to which the DR cells are resistant), checkpoint inhibitors, PARP inhibitors, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, FK506, rapamycin, mycophenolic acid, steroids, and cytokines. In yet additional aspects, the additional therapeutic agent is a checkpoint inhibitor, as described, for example, in WO2018/035413, the contents of which are expressly incorporated by reference herein. In further aspects, the additional therapeutic agent is a DDR inhibitor, including but not limited to PARP inhibitors as described, for example, in WO 2020/097306, the contents of which are expressly incorporated by reference herein.

The invention additionally encompasses a method of enhancing the cytotoxicity of a chlorotoxin (CLTX)-CAR γδ T-cells to tumor cells in a chemotherapeutic agent environment, the method comprising engineering the γδ T-cells to express at least two CLTX peptides and optionally to express a survival factor, for example, a polypeptide that confers resistance to a chemotherapeutic agent as described herein. The multivalent CLTX-CAR γδ T-cell or a composition thereof has enhanced cytotoxicity to tumor cells in the chemotherapeutic agent environment (to which the survival polypeptide confers resistance) than a comparable sCLTX-CAR γδ T-cell or composition thereof. The invention further encompasses a method of enhancing the activation (for example, CD69 activation) of a chlorotoxin (CLTX)-CAR γδ T-cells to tumor cells in a chemotherapeutic agent environment, the method comprising engineering the γδ T-cells to express at least two CLTX peptides and a survival polypeptide as described herein. The multivalent CLTX-CAR γδ T-cell or a composition thereof has enhanced activation than a comparable sCLTX-CAR γδ T-cell or composition thereof.

The combination therapies disclosed herein can be administered to patient by various routes including, for example, orally or parenterally and can include but not be limited to, intravenously, intramuscularly, subcutaneously, intraorbitally, intracapsularly, intraperitoneally, intrarectally, intracisternally, intratumorally, intravasally, intradermally, intravaginally (e.g., vaginal suppositories), or topically (e.g., powders, ointments transdermal patch) or by passive or facilitated absorption through the skin using, for example, a skin patch or transdermal iontophoresis, respectively.

Preferably, the total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose as necessary.

The pharmaceutical compositions of the invention can be formulated to be compatible with the intended method or route of administration; exemplary routes of administration are set forth herein. Furthermore, the pharmaceutical compositions can be used in combination with other therapeutically active agents or compounds as described herein in order to treat or prevent the diseases, disorders and conditions as contemplated by the present disclosure.

The pharmaceutical compositions typically comprise a therapeutically effective amount of one or more agents used in the combination therapies of the invention and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle can be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it can be stored in sterile vials as a solution, suspension, gel, emulsion, solid, or dehydrated or lyophilized powder. Such formulations can be stored either in a ready-to-use form, a lyophilized form requiring reconstitution prior to use, a liquid form requiring dilution prior to use, or other acceptable form.

Preferably, the pharmaceutical composition is provided in a single-use container (e.g., a single-use vial, ampoule, syringe, or autoinjector (similar to, e.g., an EpiPen®), whereas a multi-use container (e.g., a multi-use vial) is provided in other embodiments. Any drug delivery apparatus can be used to deliver IL-10, including implants (e.g., implantable pumps) and catheter systems, slow injection pumps and devices, all of which are well known to the skilled artisan. Depot injections, which are generally administered subcutaneously or intramuscularly, can also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions can be in the form of a sterile injectable aqueous or oleaginous suspension. This suspension can be formulated according to the known art using those suitable dispersing or wetting agents and suspending agents mentioned herein. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, CREMOPHOR EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil can be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions can be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use can be prepared according to any method known to the art for the manufacture of pharmaceutical compositions, and such compositions can contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients can be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration can be uncoated or coated by known techniques to delay disintegration and absorption in the gastrointestinal tract and thereby provide a sustained action. For example, a time-delay material such as glyceryl monostearate or glyceryl distearate can be employed. They can also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use can also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions can also contain one or more preservatives.

Oily suspensions can be formulated by suspending the active ingredient in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions can also be in the form of oil-in-water emulsions. The oily phase can be a vegetable oil, for example olive oil or arachis oil, or a mineral oil, for example, liquid paraffin, or mixtures of these. Suitable emulsifying agents can be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed.

Suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The pharmaceutical compositions suitable for use in accordance with the invention may be in any format (e.g., sprays for nasal or inhalation use) currently known or developed in the future.

The treatment methods described herein are particularly suitable for the treatment of cancer. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma is cancer that begins in the cells of the immune system.

The cancer to tumor being treated can be an intracranial tumor. Intracranial tumors include, but are not limited to, gliomas, meningiomas, acoustic neuromas, pituitary adenomas, medulloblastomas, germ cell tumors and craniopharyngiomas.

In some aspects, the cancer being treated in accordance with the invention is a CNS tumor including, but not limited to, intracranial and spinal ependymoma (excluding subependymoma); low grade infiltrative supratentorial astrocytoma/oligodendroglioma, medulloblastoma, anaplastic gliomas, glioblastoma, metastatic lesion of the CNS and primary CNS lymphoma.

In some aspects, the cancer being treated is a melanoma. Preferably the cancer being treated is uveal melanoma.

In some aspects, the cancer being treated is a neuroendocrine or adrenal tumor. Examples include but are not limited to bronchopulmonary disease, GI tract, lung or thymus, pancreas, paraganglioma or pheochromocytoma.

In some aspects, the cancer being treated is non-Hodgkin's lymphoma including but not limited to mycosis fungoides and Sezary syndrome.

In some aspects, the cancer being treated is a soft tissue sarcoma. Examples include angiosarcoma, unresectable or progressive retroperitoneal/intra-abdominal soft tissue sarcoma, rhabdomyosarcoma, extremity/superficial trunk and/or head and neck cancer, or solitary fibrous tumor/hemangiopericytoma.

In some aspects, the cancer being treated is bone cancer. Examples include Ewing's sarcoma and mesenchymal chondrosarcoma.

In some aspects, the cancer being treated is uterine sarcoma, small cell lung cancer (SCLC) or Zollinger-Ellison syndrome.

In some aspects, the cancer being treated in accordance with the invention is a gynecologic cancer (e.g., cancers of the female reproductive system) including, but not limited to ovarian cancer, cancer of the fallopian tube(s), peritoneal cancer and breast cancer. Preferably the cancer being treated in accordance with the invention is ovarian cancer.

In some aspects, a cancer being treated in accordance with the invention is glioblastoma.

Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

Also provided are kits comprising the pharmaceutical compositions typically comprise a therapeutically effective amount of one or more agents used in the combination therapies of the invention described herein. Kits typically include a label indicated the intended use of the contents of the kits and instructions for use.

Any of the compositions or a combination of the compositions described herein can be comprised in a kit. In a non-limiting example, a chimeric receptor expression construct, one or more reagents to generate a chimeric receptor expression construct, cells for transfection of the expression construct, and/or one or more instruments to obtain autologous cells for transfection of the expression construct (such an instrument may be a syringe, pipette, forceps, and/or any such medically approved apparatus). The kits may comprise one or more suitably aliquoted compositions of the present invention or reagents to generate compositions of the invention. The components of the kits may be packaged either in aqueous media or in lyophilized form. The container means of the kits may include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the chimeric receptor construct and any other reagent containers in close confinement for commercial sale. Such containers may include injection or blow molded plastic containers into which the desired vials are retained, for example.

The kits are generally in the form of a physical structure housing various components, as described below, and can be utilized, for example, in practicing the methods described above. A kit can include a composition comprising one or more of the therapeutic agents used in the combination therapy of the invention (e.g. an engineered γδ cells) provided in, e.g., one or more sterile containers, which can be in the form of a pharmaceutical composition suitable for administration to a subject. The pharmaceutical composition can be provided in a form that is ready for use or in a form requiring, for example, reconstitution or dilution prior to administration. When the compositions are in a form that needs to be reconstituted by a user, the kit can also include buffers, pharmaceutically acceptable excipients, and the like, packaged with or separately the therapeutic agent. When combination therapy is contemplated, the kit can contain the several agents separately or they can already be combined in the kit.

A kit of the invention can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via an internet site, are provided.

### EXAMPLES

The following examples are offered by way of illustration and are not to be construed as limiting the invention as claimed in any way.

### Example 1: Development of dCLTX-CAR-MGMT Vectors for γδ T-Cell Drug Resistant Immunotherapy

We have developed a novel approach to the treatment of primary GBM by combining simultaneous intracranial administration of gene-modified γδ T-cells and standard temozolomide (TMZ) maintenance chemotherapy. These γδ T-cells are transduced with O-6-Methylguanine-DNA Methyltransferase (MGMT), conveying resistance to alkylating chemotherapies, thereby allowing effector function at therapeutic concentrations of TMZ when tumor NKG2DL expression is significantly elevated. We modified γδ T-cells with a lentivector construct expressing a chimeric antigen receptor (CAR) with a chlorotoxin binding domain (CLTX-CAR) to improve GBM targeting. MGMTp140k was co-expressed within the same CLTX-CAR vector to confer TMZ resistance to the CAR-T-cells. The CLTX-CAR vectors contain a CD8α signal peptide, a mono or dual CLTX binding domain, a Myc-Tag peptide, a CD8α hinge domain, a CD28 transmembrane domain, and a costimulatory domain followed by a CD3ζ activation domain. We used P2A peptide to co-express MGMTp140k with the CAR. Initially, we demonstrated efficient transduction of the CLTX-CAR with a dual-CLTX construct as the binding domain (dCLTX-CAR) in Jurkat T-cells. Cell surface localization of the dCLTX-CAR was verified by flow cytometry. When compared with the mono-CLTX-CARs (sCLTX-CARs) transduced Jurkat-cells, the dCLTX-CARs demonstrated increased CD69 activation (MFI=4873 vs 1078). It is expected that the dCLTX-CAR-transduced γδ T cells will demonstrate greater cytotoxicity against tumor cells in vitro, including under TMZ exposure, as compared to sCLTX-CARs-transduced γδ T cells. Since γδ T-cells recognize and kill tumors through NKG2DL stress antigen recognition, we hypothesized that a CLTX-CAR without an activation signal may be sufficient to enhance recognition and cytotoxicity of γδ T-cells to tumor cells and mitigate CAR-T activation-induced cell death. We then developed dCLTX-CAR constructs without the CD3ζ domain (dCLTX-noZ-CARs). As expected, the dCLTX-noZ-CAR lentivirus transduced Jurkat-cells demonstrated enhanced cell-cell binding compared to the full dCLTX-CAR but no CD69 expression when co-cultured with GBM cells. Overall, we were able to generate dCLTX-CAR T-cells with resistance to TMZ and showed improved activation against GBM cells under TMZ exposure. Our approach of combining the dCLTX-CAR and TMZ resistance will be further validated in animal model experiments and could be a potential candidate for clinical development for GBM.

To build the dCLTX-CAR constructs, gblocks double stranded DNAs encoding human codon optimized CLTX, c-Myc Tag, P2A-EGFP, P2A-MGMTp140k and other CAR domains were synthesized by IDT DNA and cloned into transfer plasmid pDL171 by Gibson assembly cloning kit (New England Biolabs)

FIG. 3 shows flow cytometry analysis for co-expression of dCLTX-CAR and a marker gene with lentiviral vector in Jurkat cells. c-Myc tag is used as a surrogate marker of CLTX and demonstrates the expression and cell surface localization of the dCLTX-CAR. EGFP is a marker gene co-expressed with the CLTX-CAR by a P2A self-cleavage peptide.

FIG. 4 shows activation of CD69 in lentivirus dCLTX-CAR transduced Jurkat cells after co-culture with U251MG cells. Jurkat cells transduced with lentiviral vector encoding EGFP, CLTX-EGFP, dCLTX-EGFP and dCLTX-MGMT were co-cultured with U251MG cells for 24 hrs and the activation of CD69 were measured by Flow cytometry. FIG. 4 showed that T-cells transduced with a CAR comprising two CLTX peptides in the extracellular antigen-binding domain (dCLTX-CAR cells) demonstrated about 4.5 times greater CD69 activation and as compared to cells comprising a single CLTX peptides in the extracellular antigen binding domain (sCLTX-CAR γδ T-cells). While it may have been predicted that the presence of two CLTX peptides would be additive and result in greater, e.g., two-fold greater, CD69 activation than a single CLTX peptide, it was surprising that the presence of only two CLTX peptides in the CAR resulted in more than 4 times greater activation than a comparable CAR with a single CLTX peptide. This data suggested that the multiple CLTX peptides in the extracellular antigen binding domain have a synergistic effect on T cell activation and unexpectedly show greater persistence and show greater cytotoxicity against glioblastoma cells. The two CLTX peptides in the dCLTX-CAR were separated by a short peptide, specifically a Flag peptide. As shown below in FIG. 9, when a 1×CLTX construct was prepared that included a Flag or myc peptide, the 1×CLTX-CAR demonstrated higher CD69 activation than the CLTX-CAR with no peptide/tag. This data (FIG. 9; discussed in more detail below) suggests that the increased CD69 activation may be at least partially due to the addition of the peptide linker (e.g., the FLAG/myc tag).

### Example 2: Dual Chlorotoxin CAR (dCLTX-CAR/2×CLTX-CAR) and MGMT γδ-T Cells for Drug Resistant Immunotherapy of Glioblastoma Multiforme (GBM)

Flow Cytometry Assays were conducted as follows.

Activation
Lentivirus-transduced Jurkat T cells were co-cultured with U251 GBM cells for 24 hours and stained with anti-CD69 antibody

Persistence
CLTX-CAR-transduced Jurkat cells were activated and cultured for ˜3 weeks, measured % CAR+ T cells

Cytotoxicity Assay
γδ T cells transduced with high efficiency (>60%) were co-cultured with U251-GFP or U87-GFP cells at different ratios for 24-48 hours then stained with Annexin V and 7-AAD

The CLTX-CAR constructs contained either a single CLTX (1×CLTX) or multiple CLTX binding domain (e.g., 2×CLTX-CAR/dCLTX-CAR) for potential enhanced tumor targeting. Certain constructs were prepared that included a Myc-tag or Flag-tag (as indicated) for CAR-T detection/enrichment. CLTX-CAR constructs were prepared that included a CD3ζ signaling domain. In addition, CLTX-CAR constructs were also prepared without CD3ζ signaling domain (“noZ” or “without CD3z”) for mitigation of activation induced cell death (AICD) and tonic signaling. Co-expression of 06-methylguanine-DNA methyl-transferase (p140K-MGMT) to confer temozolomide (TMZ) resistance (DeltEx Drug Resistance Immunotherapy (DRI)). The data shown in FIGS. 5 to 8 represent single experiments and subsequent experiments showed similar results.

CLTX-CARs activate Jurkat T cells efficiently: Jurkat T cells were transduced with lentiviral vectors of 1× and 2×CLTX-CARs with extracellular myc or flag tags. A GFP control and a 1×CLTX-CAR without a tag were also included. FIG. 5 shows that CLTX-CAR constructs were optimized and tested in Jurkat T cells and that 2×CLTX-CARs activated Jurkat T cells efficiently. Specifically, CD69 was activated in transduced CAR-T cells but not in GFP control transduced T cells or non-transduced T cells. Further, it was observed that CLTX-CARs with an extracellular tag (myc or flag) showed higher level of T cell activation than CLTX-CAR without a tag. FIG. 9 shows that co-culture with U251 glioblastoma cells activated 1×CLTX-CAR and 2×CLTX Jurkat T cells and further that Jurkat T cells transduced with 1×CLTX-CAR or 2×CLTX-CAR constructs with no CD3z signaling domains(noZ) show no CD69 activation upon U251 co-culture. Jurkat T cells transduced with 1×CLTX-CAR with no tag show moderate activation of CD69 compared to non-transduced cells. Jurkat T cells transduced with 1×CLTX-CAR or 2×CLTX-CARs with a Flag tag show more greatly elevated CD69 activation up co-culture with tumor cells.

CLTX-CAR constructs with longer CAR-T cell persistence: Jurkat T cells were transduced with 1× and 2×CLTX-CARs with or without CD3z, activated and monitored for CAR-T percentage for about 3 weeks. FIG. 6 shows the effect of the two CLTX-CAR constructs on Jurkat T cell persistence. CLTX-CAR-T cells without CD3z have superior persistence than CLTX-CAR-T cells with CD3z. In addition, T cell persistence was improved in 2×CLTX-CARs as compared to 1×CLTX-CARs. FIG. 10 shows the effect of 2×CLTX as well as the absence of a signaling domain on Jurkat T cell persistence. Cells with no signaling domain had greater persistence than comparable cells with the CD3z signaling domain and cells with two CLTX peptides (dual CLTX) showed greater persistence than comparable cells with only one CLTX peptide. In addition, FIGS. 11 and 12 show that 1×CLTX cells that lacked a signaling domain had greater persistence than comparable cells with the CD3z signaling domain; specifically, 1×CTX-Flag-noZ-EGFP showed greater persistence than 1×CTX-Flag-Z-EGFP cells.

CLTX-CARs enhance γδ T cell killing of GBM cells: After co-culturing with CLTX-CAR-γδ T cells for 48 hrs, U251-GFP GBM cells were stained with Annexin V and 7-AAD for flow cytometric analysis of cytotoxicity. As shown in FIG. 8, over 80% of GBM cells were either undergoing apoptosis or killed when co-cultured with CLTX-CAR-γδ T cells as compared to 42.6% apoptotic GBM cells when co-cultured with non-transduced γδ T. In summary, these results indicate efficient transduction and expression of CLTX-CARs in γδ T cells and further that CLTX-CARs enhanced cytotoxicity of γδ T against GBM cells, even in the absence of a CD3z co-stimulatory domain.

### Example 3: Lentivirus Transduction of γδ Cells and Cytotoxicity of CLTX-CAR-γδ T Cells

γδ T cells with higher than 50% γδ T were expanded from healthy donor apheresis product (Hemacare) and cultured in RPMI media (Cytiva HyClone) supplemented with FBS (Cytiva HyClone), HEPES (Thermo Scientific), MEM NEAA (Cytiva HyClone), sodium pyruvate (Gibco) and human rIL-12. Expanded γδ T cells were transduced with CLTX-CAR expressing lentiviral vectors and maintained for at least 2 days before transduction efficiency analysis and cytotoxicity assays. Transduction efficiency of CLTX-γδ T cells were measured by flow cytometry gated for γδ TCR positive and Flag positive population. For cytotoxicity assays, control (γδ T cells NTC) and 2×CLTX-CAR-noZ-γδ T cells were co-cultured with U251 or U87 GBM cells in suspension at effector to target (E/T) ratio 2:1 or 4:1 for 4 hrs followed by staining with 7-AAD and flow cytometry analysis.

FIG. 13 shows microscopic pictures of control γδ T cells (γδ T cells NTC) and 2×CLTX-CAR-noZ-γδ T cells transduced with a lentiviral vector binding to GBM cells in co-culture at effector/target (E/T) of 2:1 and E/T of 4:1. CTLX-CAR γδ T cells were able to bind to GBM cells, specifically, 2×CLTX-CAR-FLAG-noZ-γδ T cells showed greater binding of GBM cells than control γδ T cells. FIG. 14 shows that 2×CLTX-CAR-noZ-γδ T showed enhanced cytotoxicity to U87 glioblastoma cells than control γδ T cells (without the CAR) even without a CD3z signaling domain.

### Example 4: Serial Killing of U251-GFP GBM Cells by 2×CLTX-CAR-noZ γδ T Cells

U251-GFP cells were established from U251MG GBM cell line transduced with GFP expressing lentiviral vector. Lentiviral transduced 2×CLTX-CAR-noZ-γδ T cells were co-cultured with U251-GFP GBM cells in a 24-well plate and time-lapse pictures were captured every one minute for 15 hours using an EVOS M5000 imaging system with EVOS onstage incubator (Thermo Scientific).

FIG. 15 show a series of still images from a time lapse movie and shows serial killing of U251MG GBM cells by 2×CLTX-CAR-noZ γδ T cells. The green cells (or as shown in gray-scale, the brighter cells) in the images are the tumor cells. The red arrows indicate the γδ T cell over time as it binds and kills different tumor cells (see, T1, T2, T3, T4 and T5 and Kill-1, Kill-2, Kill-3, Kill-4 and Kill-5 in the images). The images were taken over time (left to right, and as indicated by the arrows between the images). It shows a single 2×CLTX-CAR-noZ modified γδ T cell was able to bind, detach and eventually kill more than 5 target GBM cells within hours.

### Example 5: CARs with Three or Four CLTX Peptides are not Presented Normally on the Cell Surface

CLTX-CAR-EGFP constructs with 3 tandem CLTX peptides (3×CLTX-Z-EGFP) or 4 tandem CLTX peptides (4×CLTX-Z-EGFP) were packaged into lentiviral vector and transduced into Jurkat T cells (see, e.g., FIG. 16). 3×CLTX-Z-EGFP or 4×CLTX-Z-EGFP Jurkat T cells were co-cultured with U251 GBM cells for 24 hours and analyzed by flow cytometry.

FIGS. 17A-17C show flow cytometric analysis of cell surface staining using anti-Myc monoclonal antibody for control (NTC) cells, 3×CLTX-Z-EGFP Jurkat cells and 4×CLTX-Z-EGFP Jurkat cells and also shows any CD69 activation after co-culturing with U251 GBM cells. The cells are also gated for GFP. FIGS. 17B and 17C show that the 3×CLTX and 4×CLTX CARs are efficiently transduced with the 3× and 4×CLTX-CAR lentivectors with high GFP+ population but the CLTX-CARs are not presented normally on the cell surface (GFP+ is but not Myc+). Moreover, there is no change in CD69 activation status from those transduced Jurkat cells (GFP+), indicating no functional CAR expression on the cell surface.

While this invention has been particularly shown and described with references to preferred embodiments thereof, it will be understood by those skilled in the art that various changes in form and details may be made therein without departing from the scope of the invention encompassed by the appended claims.

1. An engineered γδ T-cell that expresses a multivalent CLTX chimeric antigen receptor (CLTX-CAR), wherein the γδ T-cells express a survival factor, wherein the survival factor is a DNA, RNA, or polypeptide that confers resistance to a chemotherapeutic agent, and further wherein:
a. the multivalent CLTX-CAR comprises:
i. an extracellular antigen-binding domain comprising at least two CLTX peptides, wherein the at least two CLTX peptides are attached by a linker peptide;
ii. a transmembrane domain; and
iii. an extracellular hinge domain that attaches the transmembrane domain to the extracellular antigen-binding domain; and
iv. optionally, an intracellular signaling domain; and
v. optionally, a co-stimulatory domain.

2. The γδ T-cell of claim 1, wherein the survival factor is a polypeptide that confers resistance to a chemotherapeutic agent.

3. The γδ T-cell of claim 2, wherein the polypeptide that confers resistance to a chemotherapeutic agent is selected from the group consisting of alkyl guanine transferase (AGT), O⁶ methylguanine DNA methyltransferase (MGMT), P140K MGMT, L22Y-DHFR, thymidylate synthase, dihydrofolate reductase, multiple drug resistance-1 protein (MDR1), 5′ nucleotidase II, dihydrofolate reductase, and thymidylate synthase.

4. The γδ T-cell of claim 3, wherein the polypeptide that confers resistance to a chemotherapeutic agent is MGMT or P140K MGMT.

5. The γδ T-cell of claim 1, wherein survival factor is a DNA that confers resistance to a chemotherapeutic agent.

6. The γδ T-cell of claim 1, wherein survival factor is an RNA that confers resistance to a chemotherapeutic agent.

7. The γδ T-cell of claim 1, wherein the transmembrane domain comprises a CD28 transmembrane domain.

8. The γδ T-cell of claim 1, wherein the peptide linker is 30 amino acids or less in length.

9. The γδ T-cell of claim 8, wherein the peptide linker is less than 15 amino acids in length.

10. The γδ T-cell of claim 1, wherein an intracellular signaling domain is present.

11. The γδ T-cell of claim 10, wherein the intracellular signaling domain comprises the CD3 zeta signaling domain.

12. The γδ T-cell of claim 1, wherein the hinge domain is the hinge region of a protein selected from the group consisting of CD8a, CD28, CD137, or a combination thereof.

13. The γδ T-cell of claim 12, wherein the hinge domain comprises the hinge region of CD8.

14. The γδ T-cell of claim 1, wherein the co-stimulatory domain is present and selected from the CD28 and 4-1BB co-stimulatory domains, or a combination thereof.

15. The γδ T-cell of claim 1, wherein the extracellular domain further comprises an extracellular signal peptide.

16. The γδ T-cell of claim 15, wherein the signal peptide is the signal peptide of a protein selected from the group consisting of CD8a, CD28, GM-CSF, CD4, CD137, or a combination thereof.

17. The γδ T-cell of claim 16, wherein the signal peptide is a CD8a signal peptide.

18. The γδ T-cell of claim 1, wherein the linker peptide is c-myc.

19. The γδ T-cell of claim 1, wherein the linker peptide is FLAG.

20. The γδ T-cell of claim 1, wherein the extracellular antigen-binding domain comprises only two CLTX peptides.

21. The γδ T-cell of claim 1, wherein the extracellular antigen-binding domain comprises only three CLTX peptides.

22. The γδ T-cell of claim 1, wherein the extracellular antigen-binding domain comprises only four CLTX peptides.

23. The γδ T-cell of claim 1, wherein the extracellular antigen-binding domain comprises only two CLTX peptides and the survival peptide is MGMT or P140K MGMT.

24. The γδ T-cell of claim 1, wherein the chemotherapeutic agent is an alkylating agent.

25. The γδ T-cell of claim 1, wherein the chemotherapeutic agent is selected from the group consisting of trimethotrexate, temozolomide, raltitrexed, S-(4-Nitrobenzyl)-6-thioinosine, 6-benzyguanidine, nitrosoureas, fotemustine, cytabarine, and camptothecin.

26. The γδ T-cell of claim 1, wherein the extracellular antigen-binding domain comprises only two CLTX peptides.

27. The γδ T-cell of claim 26, wherein the CLTX-CAR does not comprise an intracellular signaling domain.

28. The γδ T-cell of claim 26, wherein the survival factor is a polypeptide that confers resistance to a chemotherapeutic agent.

29. The γδ T-cell of claim 28, wherein the polypeptide that confers resistance to a chemotherapeutic agent is selected from the group consisting of alkyl guanine transferase (AGT), O⁶ methylguanine DNA methyltransferase (MGMT), P140K MGMT, L22Y-DHFR, thymidylate synthase, dihydrofolate reductase, multiple drug resistance-1 protein (MDR1), 5′ nucleotidase II, dihydrofolate reductase, and thymidylate synthase.

30. The γδ T-cell of claim 29, wherein the polypeptide that confers resistance to a chemotherapeutic agent is MGMT or P140K MGMT.

31. A pharmaceutical composition comprising a pharmaceutically acceptable excipient and an effective amount of the engineered γδ T-cells of claim 1.

32. (canceled)

33. (canceled)

34. A method of treating cancer or tumor in a subject in need thereof, the method comprising administering to said subject a composition comprising an effective amount of the engineered γδ T-cells of claim 1, the method further comprising co-administering to said subject the chemotherapeutic agent in an amount sufficient to increase stress antigen expression on the cancer or tumor cells.

35-51. (canceled)

52. A method of enhancing the cytotoxicity or activation of a CTX-CAR γδ T-cells to tumor cells in a subject undergoing treatment with a chemotherapeutic agent, the method comprising engineering the γδT-cells to express at least two CLTX peptides and a survival factor, wherein the survival factor is a DNA, RNA or polypeptide that confers resistance to a chemotherapeutic agent, wherein the γδ T-cell comprises a single vector that directs the expression of the CLTX-CAR and the survival factor, and further wherein:
a. the CLTX-CAR comprises:
i. an extracellular antigen-binding domain comprising at least two CLTX peptides and wherein the at least two CLTX peptides are attached by a linker peptide wherein the linker;
ii. a transmembrane domain; and
iii. an extracellular hinge domain that attaches the transmembrane domain to the extracellular antigen-binding domain;
iv. optionally, an intracellular signaling domain; and
v. optionally, a co-stimulatory domain.

53-59. (canceled)

60. A method of enhancing the persistence of CLTX-CAR γδ T-cells in a subject undergoing treatment with a chemotherapeutic agent, the method comprising engineering the γδT-cells to express at least two CLTX peptides and a survival factor, wherein the survival factor is a DNA, RNA, or polypeptide that confers resistance to a polypeptide, wherein the γδT-cell comprises a single vector that directs the expression of the CLTX-CAR and the survival factor, and further wherein:
a. the CLTX-CAR comprises:
i. an extracellular antigen-binding domain comprising at least two CLTX peptides and wherein the at least two CLTX peptides are attached by a linker peptide;
ii. a transmembrane domain; and
iii. an extracellular hinge domain that attaches the transmembrane domain to the extracellular antigen-binding domain;
iv. optionally, an intracellular signaling domain; and
v. optionally, a co-stimulatory domain.

61-83. (canceled)


SEQUENCE LISTING (15 sequences)

SEQ ID NO: 1 - protein, 36 aa, Leiurus quinquestriatus
MCMPCFTTDHQMARKCDDCCGGKGRGKCYGPQCLCR

SEQ ID NO: 2 - protein, 10 aa, Homo sapiens
EQKLISEEDL

SEQ ID NO: 3 - protein, 8 aa, Artificial Sequence
  FLAG
DYKDDDDK

SEQ ID NO: 4 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 42 aa, Homo sapiens
ALYLLRRDQRLPPDAHKPPGGGSFRTPIQEEQADAHSTLAKI

SEQ ID NO: 6 - protein, 41 aa, Homo sapiens
RSKRSRLLHSDYMNMTPRRPGPTRKHYQPYAPPRDFAAYRS

SEQ ID NO: 7 - protein, 42 aa, Homo sapiens
KRGRKKLLYIFKQPFMRPVQTTQEEDGCSCRFPEEEEGGCEL

SEQ ID NO: 8 - protein, 4 aa, Artificial Sequence
  Artificial linker
SGGG

SEQ ID NO: 9 - protein, 5 aa, Artificial Sequence
  Artificial linker
SGGGG

SEQ ID NO: 10 - protein, 4 aa, Artificial Sequence
  Artificial linker; repeated n number of times, wherein n is 1-10
SGGG

SEQ ID NO: 11 - protein, 24 aa, Homo sapiens
IYIWAPLAGTCGVLLLSLVITLYC

SEQ ID NO: 12 - protein, 27 aa, Homo sapiens
FWVLVVVGGVLACYSLLVTVAFIIFWV

SEQ ID NO: 13 - skipped

SEQ ID NO: 14 - protein, 13 aa, Influenza A virus
GLFGAIAGFIENG

SEQ ID NO: 15 - protein, 9 aa, Influenza A virus
EGMIDGWYG